SEQUENCE LISTING (23 sequences)

SEQ ID NO: 1 - protein, 6 aa, Artificial Sequence
  Synthetic Construct
HHHHHH

SEQ ID NO: 2 - protein, 11 aa, Artificial Sequence
  Synthetic Construct
YGRKKRRQRRR

SEQ ID NO: 3 - protein, 9 aa, Artificial Sequence
  Synthetic Construct
RKKRRQRRR

SEQ ID NO: 4 - protein, 26 aa, Artificial Sequence
  Synthetic Construct
ALLTAAARLLGTKNASCLVLAARHAS

SEQ ID NO: 5 - protein, 32 aa, Artificial Sequence
  Synthetic Construct
MLRAKNQLFLLSPHYLRQVKESSGSRLIQQRL

SEQ ID NO: 6 - protein, 35 aa, Artificial Sequence
  Synthetic Construct
QSWSRVYCSLAKRGHFNRISHGLQGLSAVPLRTYA

SEQ ID NO: 7 - protein, 367 aa, Artificial Sequence
  Synthetic Construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQT

SEQ ID NO: 8 - protein, 718 aa, Artificial Sequence
  Synthetic Construct
LHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 9 - protein, 4 aa, Artificial Sequence
  Synthetic Construct
MGSS

SEQ ID NO: 10 - protein, 11 aa, Artificial Sequence
  Synthetic Construct
SSGLVPRGSHM

SEQ ID NO: 11 - protein, 8 aa, Artificial Sequence
  Synthetic Construct
GSDPNSSS

SEQ ID NO: 12 - protein, 4 aa, Artificial Sequence
  Synthetic Construct
GSDP

SEQ ID NO: 13 - protein, 5 aa, Artificial Sequence
  Synthetic Construct
GSDPM

SEQ ID NO: 14 - protein, 4 aa, Artificial Sequence
  Synthetic Construct
NGIE

SEQ ID NO: 15 - protein, 7 aa, Artificial Sequence
  Synthetic Construct
ENLYFQS

SEQ ID NO: 16 - protein, 781 aa, Artificial Sequence
  Synthetic Construct
MGSSHHHHHHSSGLVPRGSHMRKKRRQRRRGSDPALLTAAARLLGTKNASCLVLAARHASGSSLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 17 - protein, 778 aa, Artificial Sequence
  Synthetic Construct
MGSSHHHHHHSSGLVPRGSHMRKKRRQRRRGSDPALLTAAARLLGTKNASCLVLAARHASLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 18 - protein, 787 aa, Artificial Sequence
  Synthetic Construct
MGSSHHHHHHSSGLVPRGSHMRKKRRQRRRGSDPNSSSLRAKNQLFLLSPHYLRQVKESSGSRLIQQRLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 19 - protein, 784 aa, Artificial Sequence
  Synthetic Construct
MGSSHHHHHHSSGLVPRGSHMRKKRRQRRRGSDPMLRAKNQLFLLSPHYLRQVKESSGSRLIQQRLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 20 - protein, 790 aa, Artificial Sequence
  Synthetic Construct
MGSSHHHHHHSSGLVPRGSHMRKKRRQRRRGSDPQSWSRVYCSLAKRGHFNRISHGLQGLSAVPLRTYAGSSLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 21 - protein, 787 aa, Artificial Sequence
  Synthetic Construct
MGSSHHHHHHSSGLVPRGSHMRKKRRQRRRGSDPQSWSRVYCSLAKRGHFNRISHGLQGLSAVPLRTYALHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 22 - protein, 1159 aa, Artificial Sequence
  Synthetic Construct
MGSSHHHHHHGSSMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNGIEENLYFQSNIRKKRRQRRRGSDPNSSSLRAKNQLFLLSPHYLRQVKESSGSRLIQQRLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 23 - protein, 756 aa, Artificial Sequence
  Synthetic Construct
MGSSHHHHHHSSGLVPRGSHMRKKRRQRRRGSDPNSSSLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

(19) United States
(12) Patent Application Publication
(10) Pub. No.: US 2019/0127724 A1
(43) Pub. Date: May 2, 2019

(54) METHYLMALONYL COENZYME A MUTASE (MCM) FUSION CONSTRUCTS FOR THE TREATMENT OF DISORDERS ASSOCIATED WITH MCM DEFICIENCY

(71) Applicant: Yissum Research Development Company of the Hebrew University Of Jerusalem Ltd., Jerusalem (IL)

(72) Inventors: Hagar Greif, Ness Ziona (IL); Anat Feldman, Jerusalem (IL); Haya Galski-Lorberboum, Jerusalem (IL)

(21) Appl. No.: 16/093,273

(22) PCT Filed: Apr. 12, 2017
(86) PCT No.: PCT/IB2017/000490

§ 371 Date: Oct. 12, 2018

Related U.S. Application Data
(60) Provisional application No. 62/321,359, filed on Apr. 12, 2016.

(51) Int. Cl. C12N 9/90

(52) U.S. Cl. CPC C12N 9/90; C12Y 504/99002; A61K 38/00; C07K 2319/10; C07K 2319/07

(57) ABSTRACT

The present invention provides compositions and methods relating to protein replacement therapy for the treatment of disorders associated with Methylmalonyl CoA Mutase.

### RELATED APPLICATIONS

This application claims the benefit of, and priority to, provisional application U.S. 62/321,359, filed Apr. 12, 2016, the contents of which are herein incorporated by reference in its entirety.

### INCORPORATION OF SEQUENCE LISTING

The contents of the text file named “BIOB008001WO SeqList.txt,” which was created on Apr. 10, 2017 and is 69 KB in size, are hereby incorporated by reference in their entirety.

### FIELD OF INVENTION

The present disclosure generally relates to Methylmalonyl CoA Mutase (MCM) fusion protein constructs that are suitable for use in enzyme replacement therapy (ERT).

### BACKGROUND

Methylmalonic Acidemia (MMA) is an autosomal recessive inherited disorder with an incidence of about 1 in every 50,000 to 100,000 people that has a poor prognosis for long-term survival.

About 60% of the MMA cases result from mutations in MUT gene leading to defects in the mitochondrial enzyme Methylmalonyl CoA mutase (MCM) that catalyzes the isomerization of methylmalonyl-CoA to succinyl-CoA and requires cobalamin (B-12) as a cofactor. Succinyl-CoA is the point of entry into the citric acid cycle (or “Krebs cycle”) for various amino acids and fats.

As known in the art, the catabolism of the branched-chain amino acids isoleucine and valine as well as methionine, threonine, odd-chain fatty acids, and cholesterol for entrance into the Krebs cycle via propionyl-coenzyme A (CoA) requires the isomerization of 1-methylmalonyl-CoA to succinyl-CoA. As indicated above, this conversion involves methylmalonyl-CoA mutase (MCM), a nuclear-encoded homodimeric apoenzyme (inactive enzyme) located in the mitochondrial matrix (1) and adenosylcobalamin, a cofactor for MCM derived from cobalamin (vitamin B-12).

The effects of MMA, which usually appear in early infancy, vary from mildto life-threatening. Affected infants can experience vomiting, dehydration, weak muscle tone (hypotonia), developmental delay, excessive tiredness (lethargy), an enlarged liver (hepatomegaly), and failure to gain weight and grow at the expected rate (failure to thrive). Long-term complications can include feeding problems, intellectual disability, chronic kidney disease, and inflammation of the pancreas (pancreatitis). Without treatment, this disorder may sometimes lead to coma and death.

Current management approaches for vitamin B-12 (hydroxocobalamin) non-responsive MMA patients include dietary restriction of propiogenic amino acids, nutritional supplement administration and vigilant monitoring. Liver or combined liver/kidney transplantations have been used to treat those with the most severe clinical manifestations (2).

Patients with MMA, even those who have received liver transplants, can develop progressive renal dysfunction (3), pathologically characterized by tubulointerstitial nephritis (4, 5), and eventually require kidney transplantation. The disease manifestations seen in the patient population, even those who have been intensively treated, demonstrate the need for new therapies, ideally ones that could target both the liver and kidney, to increase stability and protect from renal insufficiency.

Recently it was reported that rAAV9 gene therapy directed hepatic transgene expression of the Mut cDNA within 24 hours and effectively rescued the Mut−/− mice from lethality, conferred long-term survival markedly improved metabolism and resulted in striking preservation of renal function and histology. Systemic re-administration of the vector at a dose similar to that used in human clinical trials (2.5×10⁹ GC of rAAV9 per gram) to older, treated Mut−/—mice lowered circulating metabolites, increased in vivo propionate oxidative capacity and produced transgene expression in the kidney and liver (6).

There has been great progress in the use of protein translocation domain (PTD)-fusion proteins for the delivery of different macromolecules into cells in vitro and in vivo. PTDs refer to a group of short peptides that serve as delivery vectors for large molecules. Generally, PTDs are defined as short, water-soluble and partly hydrophobic, and/or polybasic peptides (at most 30-35 amino acids residues) with a net positive charge at physiological pH. PTDs are able to penetrate the cell membrane at low, micromolar concentrations in vivo and in vitro, without using any chiral receptors and without causing significant membrane damage. Furthermore, these peptides are capable of internalizing electrostatically or covalently bound biologically active cargoes, such as drugs, with high efficiency and low toxicity. This new class of peptides was introduced in the late 1980s, following the discovery of the human immunodeficiency virus type 1 (HIV-1) encoded TAT peptide and the amphiphilic Drosophila Antennapedia homeodomain-derived 16 amino acid penetration peptide (pAntp), which was discovered a few years later.

PTDs may deliver cargoes into the brain across the blood-brain barrier or target specific intracellular sub-localizations, such as the nuclei, the mitochondria and lysosomes. The ability to deliver an active enzyme into cells contributed to developing enzyme replacement therapies (ERTs), whereby the deficient or absent enzyme is artificially manufactured, purified and given to patients on a regular basis, based on the PTD delivery system.

Recently, a fusion protein for delivery of enzymes or proteins into the mitochondria was reported (7, 8). This previously reported delivery system is based on a fusion protein comprising a protein transduction domain (PTD), which facilitates the transport through both the cytoplasmic membrane and the mitochondrial membrane, fused to a mitochondrial enzyme. This fusion protein may further comprise a mitochondria targeting sequence (MTS), present between the protein transduction domain and the mitochondrial enzyme or protein. Such constructs were generally described as suitable for treatment or alleviation of a mitochondrial disorder (8).

In addition, WO 2014/170896 discloses fusion proteins comprising a PTD, MTS and a human mitochondrial protein in which the MTS is heterologous to the human mitochondrial protein present in the fusion protein construct. Such constructs were also described as suitable for treatment or alleviation of a mitochondrial disorder (9).

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic presentation of the various Methylmalonyl CoA Mutase-based (TAT-MTS-MCM) fusion proteins. Abbreviations: TAT, transactivator of transcription; MTS, mitochondrial translocation sequence; MCM, mut, methylmalonyl CoA Mutase; cs, Citrate synthase; lad, lipoamide deydrogenase; Δ, no MTS; arrow indicates Tev site; MBP, maltose binding protein.

FIG. 2 shows sodium dodecyl sulfate polyacrylamide gel electrophoresis (SDS PAGE) analyses of expression in un-induced (un), IPTG induced (in), whole cell extract (WCE) and soluble fractions (sol) of the fusion proteins TAT-MTSmcm-MCM (FIG. 2A), TAT-MTScs-MCM (FIG. 2B), TAT-MTSlad-MCM (FIG. 2C), and TAT-ΔMTS-MCM (FIG. 2D) in the various indicated bacterial hosts.

FIG. 3 shows SDS PAGE analyses of expression of the fusion protein constructs TAT-MTSmcm-MCM (FIG. 3A), TAT-MTScs-MCM (FIG. 3B), TAT-MTSlad-MCM (FIG. 3C) and TAT-AMTS-MCM (FIG. 3D) in un-induced (un), IPTG induced (in), whole cell extract (WCE) and soluble (sol) in codon+ and rosseta bacterial hosts. Western blot analyses with anti-His antibodies for the construct TAT-MTSmcm-MCM (as in FIG. 3A) is shown in FIG. 3E and for the construct TAT-MTScs-MCM (as in FIG. 3B) is shown in FIG. 3F.

FIG. 4 shows SDS PAGE analyses of the purification of the fusion protein constructs TAT-MTSmcm-MCM (FIG. 4A), TAT-MTScs-MCM (FIG. 4B), TAT-MTSlad-MCM (FIG. 4C) and TAT-AMTS-MCM (FIG. 4D) using an Ni-chelating column affinity chromatography. Abbreviations: M, marker; wce, whole cell extract; pre-run, before loading onto purification column; f1, flow through.

FIG. 5 shows a SDS PAGE analysis (FIG. 5A) and Western blot analysis using anti-MCM antibodies (FIG. 5B) for characterization of the indicated TAT-MTS-MCM fusion protein constructs. Abbreviations: HTallMUT, His-TAT-MTSmcm-MCM; HTcsMUT, His-TAT-MTScs-MCM; HTladMUT, His-TAT-MTSlad-MCM; HTΔMUT, His-TAT-ΔMTS-MCM; and MBPMUT, His-MBP-TAT-MTSmcm-MCM.

FIG. 6 shows Western blot analysis of internalization of the TAT-MTS-MCM fusion protein constructs into cells and their mitochondria. FIG. 6A shows a Western blot analysis of whole cells' extracts of 673 fibroblasts incubated with the indicated amount of the TAT-MTScs-MCM fusion protein for 24 hours, using anti-MCM antibodies. Western blot analyses of mitochondria isolated from 673 fibroblasts incubated with TAT-MTScs-MCM or TAT-MTSmcm-MCM for 3 hours using anti-His antibodies are shown in FIG. 6B and with anti-MCM antibodies are shown in FIG. 6C. FIG. 6D is a Western blot analysis of mitochondria isolated from 673 fibroblasts incubated with the various fusion protein constructs for 3 hours using anti-MCM antibodies. The fusion protein construct His-TAT-MTScs-MCM is shown at the right lane as a control. Abbreviations: Mito, mitochondria; Mito Control, mitochondria incubated without a fusion protein construct; HTallMUT, His-TAT-MTSmcm-MCM; HTcsMUT, His-TAT-MTScs-MCM; HTladMUT, His-TAT-MTSlad-MCM; HTΔMUT, His-TAT-ΔMTS-MCM; and M, marker.

FIG. 7 shows bar diagrams of relative ATP levels in patients' cells incubated with TAT-MTS-MCM fusion protein constructs. FIG. 7A-FIG. 7C show ATP levels of 346 (FIG. 7A), GM01673 (FIG. 7B) and GM00050 (FIG. 7C) fibroblasts incubated with each one of the fusion protein constructs. Fibroblasts were grown in glucose-free medium for 48 hours (15×10³ cells) and incubated with 1 μg of each of the fusion protein constructs for 6 hours and then analyzed for total cell ATP. Results represent mean±SEM, n=3, *p<0.05. Abbreviations: TAT-MTSmbp-MCM, His-MBP-TAT-MTSmcm-MCM.

FIG. 8 is a bar diagram showing the relative membrane potential (TMRE/MTG) in patients' cells incubated with TAT-MTS-MCM Fusion Protein constructs. Mitochondrial membrane potential of GM01673 fibroblasts grown in glucose-free medium for 48 hours (15×10³ cells) were incubated with 1.5 μg of the indicated fusion protein construct for 6 hours. Before the end of incubation period (1 hour), 200 nM MitoTracker Green FM was added. In addition, 30 minutes before the end of the incubation period 20 μM FCCP was added as a control and 20 minutes before the end of the incubation period 200 nM TMRE was added. Results represent mean n=2. Abbreviations: Control, cells incubated in the absence of a fusion protein construct; TMRE, tetramethylrhodamine ethyl ester; MTG, MitoTracker Green; and FCCP, Carbonyl cyanide-4-(trifluoromethoxy)phenylhydrazone.

FIG. 9 shows a bar diagram of the relative oxygen consumption of patients' cells incubated with TAT-MTS-MCM Fusion Protein constructs. Oxygen consumption of GM01673 fibroblasts grown in glucose-free medium for 48 hours (15×10³ cells) and incubated with 1 μg of the various fusion proteins for 6 hours. Oxygen consumption was determined using Seahorse Extracellular Flux (XF) Analyzer. Results represent mean±SEM, n=3, *p<0.05. Abbreviations: TAT-MTSmbp-MCM, His-MBP-TAT-MTSmcm-MCM; Control, cells incubated in the absence of the protein constructs.

FIG. 10 shows TAT-MTS-MCM fusion proteins increase cell viability of MMA patient's cells. Cell viability levels of GM01673 (A), GM00050 (B) and 346 (C) patient fibroblasts grown in glucose-free medium for 24 h. 15×103 cells were incubated with 1.5 μg of the various fusion proteins for 72 h and then analyzed for cell viability. Results represent mean±SEM, n=3, *p<0.05.

FIG. 11 shows TAT-MTS-MCM fusion proteins reduce MMA levels in MMA patient's cells. MMA ELISA analysis of 2×106 GM01673 fibroblasts grown in glucose-free medium for 24 h. Afterwards, cells were incubated with either 0.75 or 1.5 μg of TAT-MTScs-MCM or 48h and then analyzed for total cell MMA levels. Results represent mean±SEM, n=4, *p<0.05.

FIG. 12 shows TAT-MTS-MCM fusion proteins increase albumin and urea secretion from MCM (−/−) HepG2 cells. FIG. 12A-B shows secreted albumin levels in the growth medium. 5×105 HepG2 MCM (−/−) cells, confirmed to lose the MCM protein expression (A), were grown in OXPHOS dependent medium for 24h. Afterwards, cells were incubated with 1.5 μg of TAT-MTScs-MCM for 24 (FIG. 12B) or 48 h (FIG. 12C), growth medium was collected and centrifuged for 5 min 500 g. Albumin levels were determined by Western blot analysis, using anti-albumin antibodies. Equal aliquots of the growth medium were loaded. FIG. 12D shows Quantitate data of B&C. FIG. 12E shows secreted urea levels in the growth medium. 5×105 HepG2 MCM (→/→) cells were grown in complete medium supplied with 1.5 μg of TAT-MTScs-MCM for 24 h. Afterwards, cells were incubated in OXPHOS dependent medium added with 1.5 μg of TAT-MTScs-MCM for 24 h. Then growth medium was collected and centrifuged for 5 min 14000 g, the remaining cells were lysed. Urea levels were determined by Cobas analyzer. Results represent mean±SEM, n=3, *p<0.05.

FIG. 13 shows delivery of TAT-LAD into mice tissues. TAT-MTScs-MCM was injected into C57BL mice. At different time points, mice were killed, and brain, liver, and heart were removed. Tissues lysates were prepared and analyzed by Western blot analyses Using anti Methyl malonyl Co-A mutase and anti-β actin antibodies.

### DETAILED DESCRIPTION OF EMBODIMENTS

The present disclosure is based on the construction of Methylmalonyl CoA Mutase (MCM) fusion protein constructs. Such constructs comprise a protein transduction domain (TAT delivery system) and a mitochondrial translocation sequence and are suitable for use in enzyme replacement therapy (ERT) of diseases or disorders associated with a deficiency of MCM or with defective MCM.

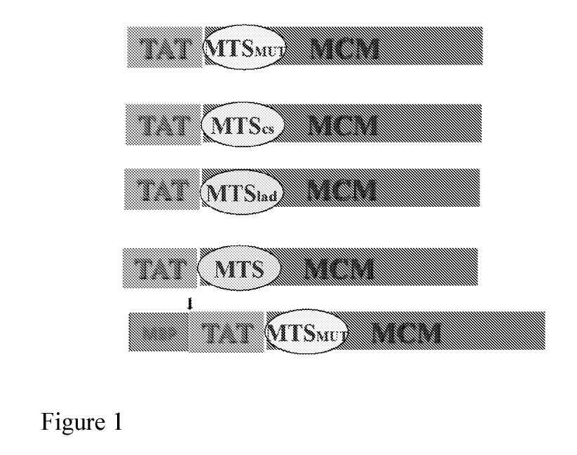

As schematically presented in FIG. 1, various MCM fusion proteins were prepared, varying in their mitochondrial translocation sequence (MTS). The MTSs used were the native MTS of Methylmalonyl CoA Mutase or an MTS of a different mitochondrial polypeptide (a heterologous polypeptide), citrate synthase (cs) and lipoamide deydrogenase (lad). As shown below, all of the fusion MCM protein constructs prepared as described herein were shown to internalize into the mitochondria and undergo the cleavage required for their activity.

Therefore by one of its aspects the present disclosure provides a fusion protein comprising a HIV-1 transactivator of transcription (TAT) domain, a functional human Methylmalonyl Coenzyme A mutase (MCM) and a human mitochondria targeting sequence (MTS) situated between said TAT domain and said functional human MCM.

The term “human Methylmalonyl Coenzyme A mutase (MCM)” refers to is an enzyme that catalyzes the isomerization of methylmalonyl-CoA to succinyl-CoA and it is involved in key metabolic pathways. It requires a vitamin B12-derived prosthetic group, adenosylcobalamin (commonly referred to as AdoCbl), to function. MCM is encoded by the nuclear MUT gene, located on chromosome 6p21 and consists of 13 exons spanning over 35 kb. The human MCM precursor contains an N-terminal mitochondrial targeting sequence (MTS) of 32 amino acids and two functional domains, a (β/α) 8 barrel (residues 88-422) substrate-binding site and a C-terminal (βα)5 B12-binding domain (residues 578-750). After entering mitochondria and removal of the leader sequence, two identical subunits form the functional enzyme (10).

The term “functional” in the context of MCM as used herein refers to any MCM polypeptide comprised in a construct as described in the present disclosure that upon entry into the mitochondria and cleavage therein is able to exert its biological activity. The biological activity of MCM may be determined according to any method known in the art, for example but not limited to as described herein below for the various fusion protein constructs.

In some specific embodiments, the functional human Methylmalonyl Coenzyme A mutase (MCM) refers to the full-length amino acid sequence of the protein.

In the above and other embodiments, the human Methylmalonyl Coenzyme A mutase (MCM) is the “mature” protein, namely refers to a protein devoid of its mitochondrial targeting sequence (MTS). In further specific embodiments MCM as herein defined has the amino acid sequence denoted by SEQ ID NO: 8.

In further embodiments, the functional human Methylmalonyl Coenzyme A mutase (MCM) is a mutated derivative of said protein, wherein one or more of the native amino acid residues of MCM has been deleted, replaced by another amino acid residue or modified while still maintaining the mitochondrial functionally of the protein.

Without wishing to be bound by theory, in all embodiments, the functional human Methylmalonyl Coenzyme A mutase (MCM) according to presently disclosed subject matter is cleaved off from the fusion protein construct upon entry to the mitochondria and resides therein at its mature, properly-folded active state. In some embodiments, upon cleavage of the MTS, functional human MCM units associate into active polypeptide dimers.

The fusion protein according to the presently disclosed subject matter may be prepared by any method known to a skilled artisan. By example, the fusion protein as herein defined may be prepared as exemplified below, by standard molecular biology and cloning techniques, by cloning the relevant nucleic acid sequences encoding the fusion protein construct into any appropriate expression vector known in the art, transforming cells with the expression vector and growing and harvesting the transformed cells to prepare the fusion protein construct. The fusion protein construct as herein defined may be then purified by methods well known to a person skilled in the art.

The term “fusion protein” in the context of the invention concerns a sequence of amino acids, predominantly (but not necessarily) connected to each other by peptidic bonds. The term “fused” in accordance with the fusion protein of the present disclosure refers to the fact that the amino acid sequences of at least three different origins, namely, the TAT domain, the sequence of the mitochondrial targeting domain (MTS) and the functional MCM, are linked to each other by covalent bonds either directly or via an amino acid linker joining (bridging, conjugating, covalently binding) the amino acid sequences. The fusion may be by chemical conjugation such as by using state of the art methodologies used for conjugating peptides.

The term “amino acid residues” as used herein refers to naturally occurring and synthetic amino acids, as well as amino acid analogs and amino acid mimetics that can function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “amino acid analogs and amino acid mimetics” refers to compounds that have the same fundamental chemical structure as a naturally occurring amino acid. Such analogs have modified R groups or modified peptide backbones, but retain the same basic chemical structure as a naturally occurring amino acid. Amino acids may be referred to herein by either their commonly known three letter symbols or by the one-letter symbols recommended by the IUPAC-IUB Biochemical Nomenclature Commission.

It is well known in the art that amino acid residues may be divided according to their chemical properties to various groups, inter alia, on the basis of similarity in polarity, charge, solubility, hydrophobicity, hydrophilicity, and/or the amphipathic nature of the residues involved.

For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q). “Basic” amino acids are selected from the group consisting of Histidine (H), lysine (K) and Arginine (R), which are polar and positively charged at pH values below their pKa's, and are very hydrophilic.

The present disclosure further relates to DNA constructs comprising the nucleic acid sequences disclosed herein. The DNA constructs of the presently disclosed subject matter may further comprise additional elements such as promoters, regulatory and control elements, translation, expression and other signals, operably linked to the nucleic acid sequence of the invention.

In some embodiments the fusion protein according to the present disclosure is wherein the functional human MCM is C-terminal to said human MTS.

Most of the proteins directed to the mitochondria are synthesized with a mitochondrial targeting (or translocation) sequence (MTS), which allows their import from the cytoplasm into mitochondria through the translocation machinery. Once entering the mitochondria, the MTS is recognized and cleaved off, allowing for proper processing and, if necessary, assembly into mitochondrial enzymatic complexes.

Thus, as used herein, the term “mitochondria targeting sequence”, “MTS” or “mitochondria translocation sequence” refers to an amino acid sequence capable of causing the transport into the mitochondria of a protein, peptide, amino acid sequence, or compound attached thereto, and any biologically active fragments thereof. MTSs used in the fusion protein constructs in accordance with the presently disclosed subject matter, which are situated N-terminal to the functional human Methylmalonyl Coenzyme A mutase (MCM), are typically from about 15 to about 40 amino acids in length, including from about 3 to about 5 nonconsecutive basic amino acid (arginine/lysine) residues, often with several serine/threonine residues but without acidic amino acid (aspartate/glutamate) residues. In their molecular structure, these MTSs are able to form strong basic amphipathic a-helices that are essential for efficient mitochondrial transportation.

As detailed herein below, in order to test the ability of the various TAT-MTS-MCM fusion protein constructs to reach the mitochondria within intact cells, patients' fibroblasts, carrying a mutation in the gene encoding for the methylmalonyl-CoA mutase (MCM) protein, were incubated in the presence of the various TAT-MTS-MCM fusion protein constructs prepared as described herein, namely TAT-MTSmcm, carrying the native MTS of MCM and TAT-MTScs-MCM and TAT-MTSlad-MCM, carrying an MTS that is heterologous to MCM, namely the MTS of citrate synthase (cs) and of lipoamide deydrogenase (lad), respectively.

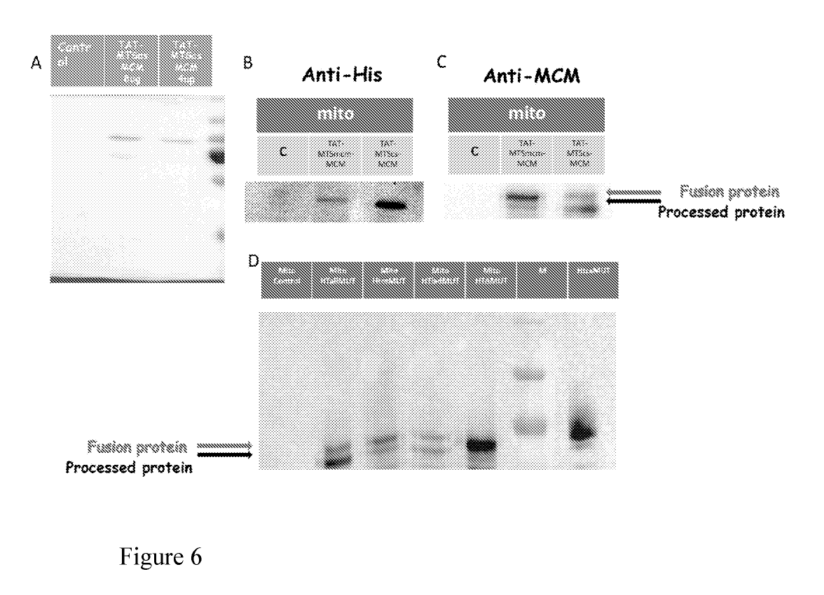

Remarkably, all of the TAT-MTS-MCM fusion protein constructs prepared as described below successfully internalized into the mitochondria and underwent processing, as demonstrated for example in FIG. 6C.

Therefore in some embodiments the fusion protein according to the present disclosure is where the human MTS is the MTS of human MCM (namely the native MTS of MCM) or heterologous to said human MCM (namely the MTS of a different mitochondrial protein/enzyme).

In specific embodiments the fusion protein according to the present disclosure is wherein the human MTS is human Methylmalonyl Coenzyme A mutase MTS, having the amino acid sequence denoted by SEQ ID NO: 5.

In still further embodiments the fusion protein according to the present disclosure is having the amino acid sequence as denoted by SEQ ID NO: 20 or SEQ ID NO: 21, both of which comprise the MTS of MCM.

As indicated above fusion protein constructs carrying an MTS that is heterologous to MCM, namely TAT-MTScs-MCM and TAT-MTSlad-MCM that include an MTS of citrate synthase (cs) and lipoamide deydrogenase (lad), respectively, were also shown to internalize into the mitochondria and undergo cleavage therein.

As used herein, the term “heterologous” when referring to MTS fused to the functional human Methylmalonyl Coenzyme A mutase (MCM) according to the present disclosure, is to be taken to mean MTS obtained from another (distinct) mitochondrial protein or enzyme, i.e. MTS which is not the naturally occurring MTS of MCM (for example but not limited to the MTS of citrate synthase or the MTS of lipoamide dehydrogenase).

Thus in some embodiments the fusion protein according to the present disclosure is wherein the human MTS is human mitochondrial citrate synthase MTS, having the amino acid sequence denoted by SEQ ID NO: 4 or human lipoamide dehydrogenase MTS, having the amino acid sequence denoted by SEQ ID NO: 6.

In specific embodiments the fusion protein according to the present disclosure is wherein the human MTS is human citrate synthase MTS having the amino acid sequence denoted by SEQ ID NO: 4.

In some embodiments the fusion protein provided by the present disclosure comprises an HIV-1 transactivator of transcription (TAT) domain having the amino acid sequence denoted by SEQ ID NO: 3 linked to functional human MCM having the amino acid sequence denoted by SEQ ID NO: 8 and a human mitochondrial citrate synthase MTS having the amino acid sequence denoted by SEQ ID NO: 4, said MTS situated between said TAT domain and said functional human MCM, and wherein said MCM is C-terminal to said MTS.

In specific embodiments the fusion protein according to the present disclosure comprising the human mitochondrial citrate synthase MTS is of the amino acid sequence denoted by SEQ ID NO: 18 or SEQ ID NO: 19.

In other specific embodiments the fusion protein according to the present disclosure is wherein the human MTS is human lipoamide dehydrogenase MTS, having the amino acid sequence denoted by SEQ ID NO: 6.

In some embodiments the fusion protein provided by the present disclosure comprises an HIV-1 transactivator of transcription (TAT) domain having the amino acid sequence denoted by SEQ ID NO. 3 linked to functional human MCM having the amino acid sequence denoted by SEQ ID NO. 3 and a human mitochondrial lipoamide dehydrogenase MTS having the amino acid sequence denoted by SEQ ID NO. 6, said MTS situated between said TAT domain and said functional human MCM, and wherein said MCM is C-terminal to said MTS.

In specific embodiments the fusion protein according to the present disclosure if having the amino acid sequence denoted by SEQ ID NO: 22 or SEQ ID NO: 23.

In some embodiments the fusion protein according to the present disclosure further comprising at least one linker. The at least one linker covalently joins different domains of the fusion protein construct.

By the term “linker” in the context of the present disclosure it is meant an amino acid sequence of from about 4 to about 20 amino acid residues positioned between the different fusion protein domains and covalently joining them together. For example, a linker in accordance with the invention may be 4, 5, 6, 7, 8, 9, 10, 11, 12, 13, 14, 15, 16, 17, 18, 19 or 20 amino acids long. Linkers are often composed of flexible amino acid residues, for example but not limited to glycine and serine so that the adjacent protein domains are free to move relative to one another. The term “linker” can be interchangeably used with “spacer”.

The design of a linker that enables proper folding of the various domains of a protein is well known in the art. Non-binding examples of a linker according to the present disclosure is any of the amino acid sequences MGSS (denoted by SEQ ID NO: 9), SSGLVPRGSHM (denoted by SEQ ID NO: 10), GSDPNSSS (denoted by SEQ ID NO: 11), GSDP (denoted by SEQ ID NO: 12), GSDPM (denoted by SEQ ID NO: 13), GSS (denoted by SEQ ID NO: 14), NGIE (denoted by SEQ ID NO: 15), NI (denoted by SEQ ID NO: 16).

The fusion protein in the context of the invention may also optionally comprise at least one methionine (M) residue at its N-terminus, as in the case of the exemplified fusion proteins below. The methionine is positioned N-terminal to the TAT domain.

Fusion may also be achieved by recombinant techniques, i.e. by construction of a nucleic acid sequence coding for the entire the fusion protein (coding for all segments) so that essentially all the bonds are peptidic bonds.

In order to facilitate purification of the protein constructs described herein, fusion protein constructs in accordance with the present disclosure may also comprise an N-terminal tag (e.g. His tag as exemplified below, Glutathione S-transferase (GST), Maltose-Binding Protein (MBP), FLAG octapeptide, to name but few), which may be removed or retained in the final fusion construct. Such tags are normally cleaved off from the fusion protein upon entry to the mitochondria, along with the TAT and MTS sequences.

Thus in some embodiments the fusion protein according to the present disclosure further comprises at least one purification tag (in order to facilitate purification, e.g. a His tag or a maltose-binding protein (MBP) tag).

The purification tag may also be cleaved off from the fusion protein construct according to the present disclosure by inserting a protease cleavage site at an appropriate site in the vicinity of the purification tag. Therefore in some embodiments the fusion protein according to the present disclosure further comprised at least one protease cleavage site.

For example, the fusion protein construct TAT-MTScs-MCM denoted by SEQ ID NO: 18 comprises from its N to C termini a linker having the amino acid sequence MGSS (denoted by SEQ ID NO: 9), a histidine tag (having the amino acid sequence HHHHHH, denoted by SEQ ID NO: 1), an additional linker having the amino acid sequence SSGLVPRGSHM (denoted by SEQ ID NO: 10), a TAT domain (having the amino acid sequence RKKRRQRRR, denoted by SEQ ID NO: 3), a further linker having the amino acid sequence GSDP (denoted by SEQ ID NO: 12), the MTS of citrate synthase (denoted by SEQ ID NO: 4), an additional linker situated between the MTS and MCM having the amino acid sequence of GSS (denoted by SEQ ID NO: 14) and the MCM protein (denoted by SEQ ID NO: 8).

Therefore in some embodiments the fusion protein according to the present disclosure is wherein the MTS is linked to said functional MCM and/or to said TAT via a linker.

As indicated above, the fusion protein construct as herein defined also comprise HIV-1 transactivator of transcription (TAT) domain. As used herein, the term “HIV-1 transactivator of transcription (TAT)” domain refers to a portion of a protein that is encoded by the tat gene in HIV-1, which is an 11-amino-acid arginine- and lysine-rich portion of the HIV-1 Tat protein. In some embodiments TAT as herein described is having the amino acid sequence YGRKKRRQRRR as set forth in SEQ ID NO. 2.

The presently disclosed subject matter also encompasses any fragments of the above defined TAT domain. For example, a TAT domain according to the presently disclosed subject matter may comprise from about 3 to about 11 (e.g. 4-11, 5-11, 6-11, 7-11, 8-11, 9, 10 or 11) sequential amino acid residues of the HIV-1 Tat protein having the amino acid sequence YGRKKRRQRRR (SEQ ID NO. 2).

In some embodiments, the fragment of the above defined TAT domain comprise 9 sequential amino acid residues of the HIV-1 Tat protein, having the amino acid sequence of RKKRRQRRR, as set forth in SEQ ID NO. 3 as used in the preparation of the fusion protein constructs exemplified below.

Thus, in the above and other embodiments of the presently disclosed subject matter, the fusion protein comprises a TAT domain at its N-terminus and a functional MCM at its C-terminus, both covalently linked (fused) to an MTS that is situated between said TAT domain and said functional MCM. In other words, the disclosure provides a protein construct comprising an N-terminal TAT fused to N-terminal of MTS fused to N-terminal of functional MCM, as schematically presented in FIG. 1.

By another one of its aspects the present disclosure provides a composition comprising a physiologically acceptable carrier and as an active ingredient a fusion protein as herein defined.

By still another one of its aspects the present disclosure provides a pharmaceutical composition comprising a pharmaceutically acceptable carrier and as an active ingredient a fusion protein as herein defined.

The “composition” as herein defined generally comprises a buffering agent, an agent which adjusts the osmolarity thereof, and optionally, one or more pharmaceutically (or physiologically) acceptable carriers, diluents, additives and excipients as known in the art. Supplementary active ingredients can also be incorporated into the compositions. The pharmaceutically acceptable carrier can be solvent or dispersion medium containing, for example, water, ethanol, polyol (for example, glycerol, propylene glycol, and liquid polyethylene glycol, and the like), suitable mixtures thereof, and vegetable oils. Each carrier should be physiologically or pharmaceutically acceptable, as the case may be, in the sense of being compatible with the other ingredients and not injurious to the patient.

The additives may be but are not limited to at least one of a protease inhibitor, for example phenylmethanesulfonylfluoride or phenylmethylsulfonyl fluoride (PMSF), Nafamostat Mesylate, 4-(2-Aminoethyl)benzenesulfonyl fluoride hydrochloride (AEBSF), Bestatin, Pepstatin A, E-64, Leupeptin, 1,10-Phenanthroline and any other protease inhibitor known in the art.

The “pharmaceutical compositions” of the presently disclosed subject matter are compositions as described above, comprising pharmaceutically acceptable carriers, diluent, adjuvant and/or excipients and/or additives as known in the art.

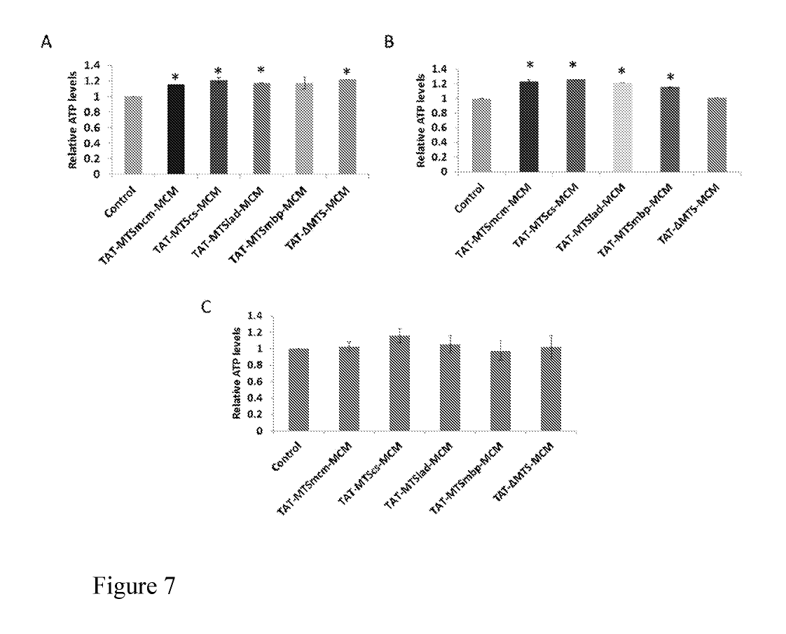

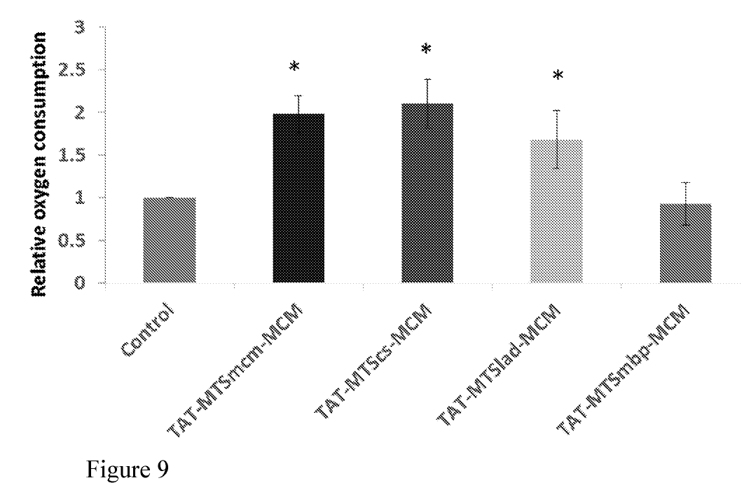

As detailed below, the various mitochondrial-targeted MCM fusion protein constructs prepared as described herein internalize into the mitochondria and undergo cleavage into their active form. This was demonstrated in vitro via the ability of the fusion protein constructs to affect ATP produced by oxidative phosphorylation (OXPHOS) in the mitochondria of GM01673 cells obtained from MMA patients. As shown in FIG. 7B, an increase of 15-25% was observed in ATP levels upon treatment with the various fusion protein constructs, while the fusion protein construct TAT-MTScs-MCM resulted in the largest increase. In addition, as shown in FIG. 9, the mitochondrial targeted MCM fusion protein constructs were shown in GM01673 fibroblasts to affect oxygen consumption by the mitochondria, which is an additional marker of mitochondrial activity (FIG. 9).

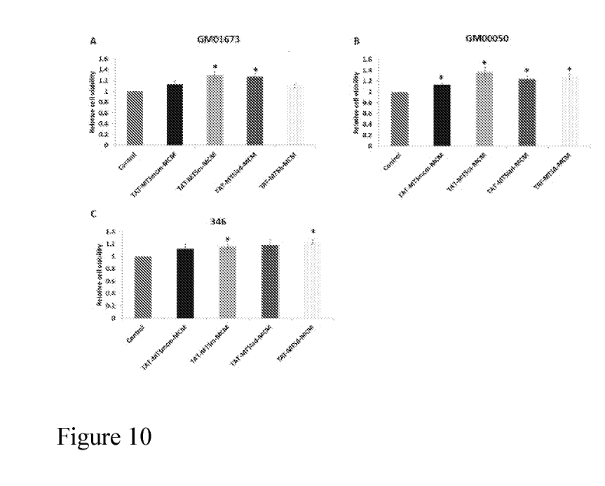

Furthermore, the mitochondrial targeted MCM fusion protein constructs were also shown to affect cell viability in GM01673 cells from methylmalonic acidemia (MMA) patients. As shown in FIG. 10, a significant enhancement in cell viability relative to the control was observed in GM01673 fibroblasts with TAT-MTScs-MCM (27%) and TAT-MTSlad-MCM (24%) fusion protein constructs.

Therefore by still another aspect, the present disclosure provides a pharmaceutical composition as herein defined for treating or alleviating a disease or disorder associated with a deficiency of MCM or with defective MCM.

As known in the art and as used herein the term “disease or disorder associated with a deficiency of MCM or with defective MCM” refers to any disease, disorder, condition or illness that affects a subject having a deficiency of MCM or defective MCM. Deficiency of MCM or defective MCM may arise from, but are not limited to, mutations in the MUT gene encoding MCM.

The term “disease or disorder associated with a deficiency of MCM or with defective MCM” encompasses for example methylmalonic acidemia (MMA).

In the above and other embodiments the disease or disorder associated with a deficiency of MCM or with defective MCM as herein defined is methylmalonic acidemia (MMA).

The term “methylmalonic acidemia” (MMA) as known in the art and as used herein refers to an autosomal recessive inherited disorder resulting from mutations in the mitochondrial enzyme Methylmalonyl CoA mutase (MCM) that catalyzes the isomerization of methylmalonyl-CoA to succinyl-CoA and requires cobalamin (B12) as a cofactor. Therefore deficiency in either MCM or adenosylcobalamin causes methylmalonic acidemia.

MMA encompasses isolated methylmalonic acidemia (OMIM 251000) and Methylmalonic acidemia and homocystinuria, OMIM 277400.

Therefore in some embodiments of the present disclosure MMA is isolated MMA, OMIM 251000. “Isolated methylmalonic acidemia” also known as mut-type MMA (OMIM 251000) as known in the art and as herein defined is an autosomal recessive disorder, commonly presents with metabolic acidosis and hyperammonemia. The Age at onset of symptoms and the in vivo response to cobalamin are the strongest predictors of disease course and survival. Genotype-phenotype correlations have been limited due to the mixture and abundance of both homozygous and compound heterozygous mutations, particularly in the MUT gene that encodes MCM (11). The mut-type MMA also referred to as isolated methylmalonic acidemia (OMIM 251000) is caused by a defect in MCM apoenzyme, which is encoded by the nuclear MUT gene.

The effects of MMA, which usually appear in early infancy, vary from mild to life-threatening. Affected infants can experience vomiting, dehydration, weak muscle tone (hypotonia), developmental delay, excessive tiredness (lethargy), an enlarged liver (hepatomegaly), and failure to gain weight and grow at the expected rate (failure to thrive). Long-term complications can include feeding problems, intellectual disability, chronic kidney disease, and inflammation of the pancreas (pancreatitis). Without treatment, this disorder may sometimes lead to coma and death.

Current management approaches for vitamin B12 (hydroxocobalamin) non-responsive MMA patients include dietary restriction of propiogenic amino acids, nutritional supplement administration and vigilant monitoring. Liver or combined liver/kidney transplantations have been used to treat those with the most severe clinical manifestations.

Patients with MMA, even those who have received liver transplants, can develop progressive renal dysfunction and may eventually require kidney transplantation. The disease manifestations seen in the patient population, even those who have been intensively treated, demonstrate the need for new therapies, ideally ones that could target both the liver and kidney, to increase stability and protect from renal insufficiency.

The present disclosure further provides the fusion protein or the pharmaceutical composition as herein defined for use in a method of treatment or alleviation of a disease or disorder associated with a deficiency of MCM or with defective MCM.

Still further the present disclosure provides a method for treating or alleviating a disease or disorder associated with a deficiency of MCM or with defective MCM in a subject in need thereof, said method comprising the step of administering to said subject a therapeutically effective amount of the fusion protein accordin or the pharmaceutical composition according to the present disclosure, thereby treating or alleviating a disease or disorder associated with a deficiency of MCM or with defective MCM.

The term “treat”, “treatment”, “alleviating” or forms thereof as herein defined means to prevent worsening or arrest or alleviate or cure the disease or condition in a subject in need thereof, namely a disease or condition associated with a deficiency of MCM or with defective MCM, e.g. methylmalonic acidemia (MMA).

The term “treatment”, “treating” or “alleviating” in the context of the intention does not refers to complete curing of the disease(s), as it does not change the mutated genetics causing the disease. This term refers to alleviating at least one of the undesired symptoms associated with the disease, improving the quality of life of the subject, decreasing disease-caused mortality, or (if the treatment in administered early enough) preventing the full manifestation of the mitochondrial disorder before it occurs, mainly to organs and tissues that have a high energy demand.

In other words the present disclosure provides a fusion protein or a pharmaceutical composition as herein defined or a method comprising administering said fusion protein or pharmaceutical composition for substituting, at least in part, activity of a defective, deficient or non-functional human MCM in a subject in need.

Therefore by still another aspect the present disclosure provides a method for substituting, at least in part, activity of a defective, deficient or non-functional human MCM in a subject in need, comprising administering to said subject a therapeutically effective amount of the fusion protein or the pharmaceutical composition as herein defined.

By the term “substituting” as used herein it is meant to replace, exchange, use instead of, or use as an alternative to a defective or non-functional MCM.

In some embodiments the functional human MCM protein substitutes, at least in part, activity of a defective, deficient or non-functional human MCM in a subject in need. In specific embodiments the functional human MCM protein provides in a subject in need at least 5 percent, at least 10 percent, at least 20 percent, at least 30 percent, at least 40 percent, at least 50 percent, at least 60 percent, at least 70 percent, at least 80 percent, at least 90 percent or up to 100 percent of the activity of a non-defective human MCM.

The present disclosure further provides a method for introducing a functional human Methylmalonyl Coenzyme A mutase (MCM) protein into the mitochondria of a subject in need thereof, said method comprising the step of administering to said subject a therapeutically effective amount of the fusion protein or the pharmaceutical composition according to the present disclosure, thereby introducing a functional human MCM protein into the mitochondria of said subject.

Therapeutic compositions (or formulations) or pharmaceutical compositions may be administered in any conventional route and dosage as determined by a person skilled in the art. Administration can be any one of intravenous, intraperitoneal, intramuscular and intrathecal administration. Oral administration is also contemplated.

In specific embodiments the fusion protein or pharmaceutical composition as herein defined is intravenously administered to said subject.

The term “therapeutically effective amount” (or amounts) of the fusion peptide according to the present disclosure for purposes herein defined is determined by such considerations as are known in the art in order to cure or at least arrest or at least alleviate the medical condition.

In some embodiments the therapeutically effective amount may be determined for each patient individually, based on the patient's basal protein activity of MCM. The patient's basal protein activity or the level of protein activity may in turn be determined using any method known in the art.

In some embodiments the method according to the present disclosure further comprises administering to said subject an additional therapeutic agent.

The term “additional therapeutic agent” in the context of a disease or disorder associated with a deficiency of MCM or with defective MCM (e.g. MMA disorder(s)) are any standard of care therapy known to a person skilled in the art, for example dietary restriction of propiogenic amino acids and nutritional supplement administration.

By the term “subject” as used herein it is meant any warm-blooded animals, such as for example rats, mice, dogs, cats, guinea pigs, primates and humans for which administration of the therapeutic agent as herein defined, or any pharmaceutical composition of the invention is desired, namely a subject diagnosed as having a disease or disorder associated with a deficiency of MCM or with defective MCM (e.g. MMA).

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range.

It is appreciated that certain features of the presently disclosed subject matter, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Unless defined otherwise, all technical and scientific terms used herein have the same meaning as commonly understood by one of ordinary skill in the art to which this invention is related. The following terms are defined for purposes of the invention as described herein.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook & Russell, 2001.

### Example 1

### General Methods

### Cell Culture

Fibroblasts (GM01673, GM00050) from methylmalonic acidemia (MMA) patients were obtained from Coriell Cell Repositories (Camden, N.J.) and grown in the recommended medium (MEM eagle RPMI medium supplemented with 10% hyclone FBS, 2 mM L-glutamine, 100 U/mL penicillin, and 100 μg/mL streptomycin, Biological Industries, Beit Ha'emek, Israel). 346 fibroblasts were obtained from the Department of Genetic and Metabolic Diseases, Hadassah medical center, and grown in the same medium as above. All cell lines were grown at 37° C. in humidified atmosphere of 5% CO₂.

### Plasmid Construction

TABLE 1
Amino acid sequences of the fusion protein constructs and their
components

| SEQ ID | | |
|---|---|---|
| NO. | Amino acid sequence | Description |
| 1 | HHHHHH | His tag |
| 2 | YGRKKRRQRRR | TAT |
| 3 | RKKRRQRRR | TAT fragment |
| 4 | ALLTAAARLLGTKNASCLVLAARHAS | MTS of citrate |
| | | synthase |
| | | (MTScs) |
| 5 | MLRAKNQLFLLSPHYLRQVKESSGSRLIQQRL | MTS of |
| | | Methylmalonyl |
| | | CoA mutase |
| | | (MTSmcm) |
| 6 | QSWSRVYCSLAKRGHFNRISHGLQGLSAVPLRT | MTS of |
| | YA | lipoamide |
| | | deydrogenase |
| | | (MTSlad) |
| 7 | MKIEEGKLVIWINGDKGYNGLAEVGKKFEKDTG | MBP |
| | IKVTVEHPDKLEEKFPQVAATGDGPDIIFWAHDR | |
| | FGGYAQSGLLAEITPDKAFQDKLYPFTWDAVRY | |
| | NGKLIAYPIAVEALSLIYNKDLLPNPPKTWEEIPA | |
| | LDKELKAKGKSALMFNLQEPYFTWPLIAADGGY | |
| | AFKYENGKYDIKDVGVDNAGAKAGLTFLVDLI | |
| | KNKHMNADTDYSIAEAAFNKGETAMTINGPWA | |
| | WSNIDTSKVNYGVTVLPTFKGQPSKPFVGVLSA | |
| | GINAASPNKELAKEFLENYLLTDEGLEAVNKDK | |
| | PLGAVALKSYEEELAKDPRIAATMENAQKGEIM | |
| | PNIPQMSAFWYAVRTAVINAASGRQTVDEALKD | |
| | AQT | |
| 8 | LHQQQPLHPEWAALAKKQLKGKNPEDLIWHTP | Methylmalonyl |
| | EGISIKPLYSKRDTMDLPEELPGVKPFTRGPYPT | CoA mutase |
| | MYTFRPWTIRQYAGFSTVEESNKFYKDNIKAG | (MCM) |
| | QQGLSVAFDLATHRGYDSDNPRVRGDVGMA | |
| | GVAIDTVEDTKILFDGIPLEKMSVSMTMNGAVIP | |
| | VLANFIVTGEEQGVPKEKLTGTIQNDILKEFMVR | |
| | NTYIFPPEPSMKIIADIFEYTAKHMPKFNSISISGY | |
| | HMQEAGADAILELAYTLADGLEYSRTGLQAGLT | |
| | IDEFAPRLSFFWGIGMNFYMEIAKMRAGRRLWA | |
| | HLIEKMFQPKNSKSLLLRAHCQTSGWSLTEQDP | |
| | YNNIVRTAIEAMAAVFGGTQSLHTNSFDEALGL | |
| | PTVKSARIARNTQIIIQEESGIPKVADPWGGSYM | |
| | MECLTNDVYDAALKLINEIEEMGGMAKAVAEGI | |
| | PKLRIEECAARRQARIDSGSEVIVGVNKYQLEKE | |
| | DAVEVLAIDNTSVRNRQIEKLKKIKSSRDQALAE | |
| | RCLAALTECAASGDGNILALAVDASRARCTVGE | |
| | ITDALKKVFGEHKANDRMVSGAYRQEFGESKEI | |
| | TSAIKRVHKFMEREGRRPRLLVAKMGQDGHDR | |
| | GAKVIATGFADLGFDVDIGPLFQTPREVAQQAV | |
| | DADVHAVGISTLAAGHKTLVPELIKELNSLGRPD | |
| | ILVMCGGVIPPQDYEFLFEVGVSNVFGPGTRIPK | |
| | AAVQVLDDIEKCLEKKQQSV | |
| 9 | MGSS | Linker 1 |
| 10 | SSGLVPRGSHM | Linker 2 |
| 11 | GSDPNSSS | Linker 3 |
| 12 | GSDP | Linker 4 |
| 13 | GSDPM | Linker 5 |
| N/A | GSS | Linker 6 |
| 15 | NGIE | Linker 7 |
| N/A | NI | Linker 8 |
| 17 | ENLYFQS | TEV site |
| 18 | MGSSHHHHHHSSGLVPRGSHMRKKRRQRRRGS | TAT-MTScs- |
| | DPALLTAAARLLGTKNASCLVLAARHASGSSLH | MCM |
| | QQQPLHPEWAALAKKQLKGKNPEDLIWHTPEGI | |
| | SIKPLYSKRDTMDLPEELPGVKPFTRGPYPTMYT | |
| | FRPWTIRQYAGFSTVEESNKFYKDNIKAGQQGL | |
| | SVAFDLATHRGYDSDNPRVRGDVGMAGVAIDT | |
| | VEDTKILFDGIPLEKMSVSMTMNGAVIPVLANFI | |
| | VTGEEQGVPKEKLTGTIQNDILKEFMVRNTYIFP | |
| | PEPSMKIIADIFEYTAKHMPKFNSISISGYHMQEA | |
| | GADAILELAYTLADGLEYSRTGLQAGLTIDEFAP | |
| | RLSFFWGIGMNFYMEIAKMRAGRRLWAHLIEK | |
| | MFQPKNSKSLLLRAHCQTSGWSLTEQDPYNNIV | |
| | RTAIEAMAAVFGGTQSLHTNSFDEALGLPTVKS | |
| | ARIARNTQIIIQEESGIPKVADPWGGSYMMECLT | |
| | NDVYDAALKLINEIEEMGGMAKAVAEGIPKLRI | |
| | EECAARRQARIDSGSEVIVGVNKYQLEKEDAVE | |
| | VLAIDNTSVRNRQIEKLKKIKSSRDQALAERCLA | |
| | ALTECAASGDGNILALAVDASRARCTVGEITDA | |
| | LKKVFGEHKANDRMVSGAYRQEFGESKEITSAI | |
| | KRVHKFMEREGRRPRLLVAKMGQDGHDRGAK | |
| | VIATGFADLGFDVDIGPLFQTPREVAQQAVDAD | |
| | VHAVGISTLAAGHKTLVPELIKELNSLGRPDILV | |
| | MCGGVIPPQDYEFLFEVGVSNVFGPGTRIPKAAV | |
| | QVLDDIEKCLEKKQQSV | |
| 19 | MGSSHHHHHHSSGLVPRGSHMRKKRRQRRRGS | TAT-MTScs- |
| | DPALLTAAARLLGTKNASCLVLAARHASLHQQ | MCM variant |
| | QPLHPEWAALAKKQLKGKNPEDLIWHTPEGISIK | |
| | PLYSKRDTMDLPEELPGVKPFTRGPYPTMYTFRP | |
| | WTIRQYAGFSTVEESNKFYKDNIKAGQQGLSVA | |
| | FDLATHRGYDSDNPRVRGDVGMAGVAIDTVED | |
| | TKILFDGIPLEKMSVSMTMNGAVIPVLANFIVTG | |
| | EEQGVPKEKLTGTIQNDILKEFMVRNTYIFPPEPS | |
| | MKIIADIFEYTAKHMPKFNSISISGYHMQEAGAD | |
| | AILELAYTLADGLEYSRTGLQAGLTIDEFAPRLSF | |
| | FWGIGMNFYMEIAKMRAGRRLWAHLIEKMFQP | |
| | KNSKSLLLRAHCQTSGWSLTEQDPYNNIVRTAIE | |
| | AMAAVFGGTQSLHTNSFDEALGLPTVKSARIAR | |
| | NTQIIIQEESGIPKVADPWGGSYMMECLTNDVY | |
| | DAALKLINEIEEMGGMAKAVAEGIPKLRIEECAA | |
| | RRQARIDSGSEVIVGVNKYQLEKEDAVEVLAID | |
| | NTSVRNRQIEKLKKIKSSRDQALAERCLAALTEC | |
| | AASGDGNILALAVDASRARCTVGEITDALKKVF | |
| | GEHKANDRMVSGAYRQEFGESKEITSAIKRVHK | |
| | FMEREGRRPRLLVAKMGQDGHDRGAKVIATGF | |
| | ADLGFDVDIGPLFQTPREVAQQAVDADVHAVG | |
| | ISTLAAGHKTLVPELIKELNSLGRPDILVMCGGVI | |
| | PPQDYEFLFEVGVSNVFGPGTRIPKAAVQVLDDI | |
| | EKCLEKKQQSV | |
| 20 | MGSSHHHHHHSSGLVPRGSHMRKKRRQRRRGS | TAT-MTSmcm- |
| | DPNSSSLRAKNQLFLLSPHYLRQVKESSGSRLIQ | MCM |
| | QRLLHQQQPLHPEWAALAKKQLKGKNPEDLIW | |
| | HTPEGISIKPLYSKRDTMDLPEELPGVKPFTRGPY | |
| | PTMYTFRPWTIRQYAGFSTVEESNKFYKDNIKA | |
| | GQQGLSVAFDLATHRGYDSDNPRVRGDVGMAG | |
| | VAIDTVEDTKILFDGIPLEKMSVSMTMNGAVIPV | |
| | LANFIVTGEEQGVPKEKLTGTIQNDILKEFMVRN | |
| | TYIFPPEPSMKIIADIFEYTAKHMPKFNSISISGYH | |
| | MQEAGADAILELAYTLADGLEYSRTGLQAGLTI | |
| | DEFAPRLSFFWGIGMNFYMEIAKMRAGRRLWA | |
| | HLIEKMFQPKNSKSLLLRAHCQTSGWSLTEQDP | |
| | YNNIVRTAIEAMAAVFGGTQSLHTNSFDEALGL | |
| | PTVKSARIARNTQIIIQEESGIPKVADPWGGSYM | |
| | MECLTNDVYDAALKLINEIEEMGGMAKAVAEGI | |
| | PKLRIEECAARRQARIDSGSEVIVGVNKYQLEKE | |
| | DAVEVLAIDNTSVRNRQIEKLKKIKSSRDQALAE | |
| | RCLAALTECAASGDGNILALAVDASRARCTVGE | |
| | ITDALKKVFGEHKANDRMVSGAYRQEFGESKEI | |
| | TSAIKRVHKFMEREGRRPRLLVAKMGQDGHDR | |
| | GAKVIATGFADLGFDVDIGPLFQTPREVAQQAV | |
| | DADVHAVGISTLAAGHKTLVPELIKELNSLGRPD | |
| | ILVMCGGVIPPQDYEFLFEVGVSNVFGPGTRIPK | |
| | AAVQVLDDIEKCLEKKQQSV | |
| 21 | MGSSHHHHHHSSGLVPRGSHMRKKRRQRRRGS | TAT-MTSmcm- |
| | DPMLRAKNQLFLLSPHYLRQVKESSGSRLIQQRL | MCM |
| | LHQQQPLHPEWAALAKKQLKGKNPEDLIWHTP | Variant |
| | EGISIKPLYSKRDTMDLPEELPGVKPFTRGPYPT | |
| | MYTFRPWTIRQYAGFSTVEESNKFYKDNIKAGQ | |
| | QGLSVAFDLATHRGYDSDNPRVRGDVGMAGVA | |
| | IDTVEDTKILFDGIPLEKMSVSMTMNGAVIPVLA | |
| | NFIVTGEEQGVPKEKLTGTIQNDILKEFMVRNTY | |
| | IFPPEPSMKIIADIFEYTAKHMPKFNSISISGYHMQ | |
| | EAGADAILELAYTLADGLEYSRTGLQAGLTIDE | |
| | FAPRLSFFWGIGMNFYMEIAKMRAGRRLWAHLI | |
| | EKMFQPKNSKSLLLRAHCQTSGWSLTEQDPYNN | |
| | IVRTAIEAMAAVFGGTQSLHTNSFDEALGLPTVK | |
| | SARIARNTQIIIQEESGIPKVADPWGGSYMMECL | |
| | TNDVYDAALKLINEIEEMGGMAKAVAEGIPKLR | |
| | IEECAARRQARIDSGSEVIVGVNKYQLEKEDAV | |
| | EVLAIDNTSVRNRQIEKLKKIKSSRDQALAERCL | |
| | AALTECAASGDGNILALAVDASRARCTVGEITD | |
| | ALKKVFGEHKANDRMVSGAYRQEFGESKEITSA | |
| | IKRVHKFMEREGRRPRLLVAKMGQDGHDRGAK | |
| | VIATGFADLGFDVDIGPLFQTPREVAQQAVDAD | |
| | VHAVGISTLAAGHKTLVPELIKELNSLGRPDILV | |
| | MCGGVIPPQDYEFLFEVGVSNVFGPGTRIPKAAV | |
| | QVLDDIEKCLEKKQQSV | |
| 22 | MGSSHHHHHEISSGLVPRGSHMRKKRRQRRRGS | TAT-MTSlad- |
| | DPQSWSRVYCSLAKRGHFNRISHGLQGLSAVPL | MCM |
| | RTYAGSSLHQQQPLHPEWAALAKKQLKGKNPE | |
| | DLIWHTPEGISIKPLYSKRDTMDLPEELPGVKPFT | |
| | RGPYPTMYTFRPWTIRQYAGFSTVEESNKFYKD | |
| | NIKAGQQGLSVAFDLATHRGYDSDNPRVRGDV | |
| | GMAGVAIDTVEDTKILFDGIPLEKMSVSMTMNG | |
| | AVIPVLANFIVTGEEQGVPKEKLTGTIQNDILKEF | |
| | MVRNTYIFPPEPSMKIIADIFEYTAKHMPKFNSISI | |
| | SGYHMQEAGADAILELAYTLADGLEYSRTGLQA | |
| | GLTIDEFAPRLSFFWGIGMNFYMEIAKMRAGRR | |
| | LWAHLIEKMFQPKNSKSLLLRAHCQTSGWSLTE | |
| | QDPYNNIVRTAIEAMAAVFGGTQSLHTNSFDEA | |
| | LGLPTVKSARIARNTQIIIQEESGIPKVADPWGGS | |
| | YMMECLTNDVYDAALKLINEIEEMGGMAKAVA | |
| | EGIPKLRIEECAARRQARIDSGSEVIVGVNKYQL | |
| | EKEDAVEVLAIDNTSVRNRQIEKLKKIKSSRDQA | |
| | LAERCLAALTECAASGDGNILALAVDASRARCT | |
| | VGEITDALKKVFGEHKANDRMVSGAYRQEFGE | |
| | SKEITSAIKRVHKFMEREGRRPRLLVAKMGQDG | |
| | HDRGAKVIATGFADLGFDVDIGPLFQTPREVAQ | |
| | QAVDADVHAVGISTLAAGHKTLVPELIKELNSL | |
| | GRPDILVMCGGVIPPQDYEFLFEVGVSNVFGPGT | |
| | RIPKAAVQVLDDIEKCLEKKQQSV | |
| 23 | MGSSHHHHHHSSGLVPRGSHMRKKRRQRRRGS | TAT-MTSlad- |
| | DPQSWSRVYCSLAKRGHFNRISHGLQGLSAVPL | MCM |
| | RTYALHQQQPLHPEWAALAKKQLKGKNPEDLI | Variant |
| | WHTPEGISIKPLYSKRDTMDLPEELPGVKPFTRG | |
| | PYPTMYTFRPWTIRQYAGFSTVEESNKFYKDNIK | |
| | AGQQGLSVAFDLATHRGYDSDNPRVRGDVGMA | |
| | GVAIDTVEDTKILFDGIPLEKMSVSMTMNGAVIP | |
| | VLANFIVTGEEQGVPKEKLTGTIQNDILKEFMVR | |
| | NTYIFPPEPSMKIIADIFEYTAKHMPKFNSISISGY | |
| | HMQEAGADAILELAYTLADGLEYSRTGLQAGLT | |
| | IDEFAPRLSFFWGIGMNFYMEIAKMRAGRRLWA | |
| | HLIEKMFQPKNSKSLLLRAHCQTSGWSLTEQDP | |
| | YNNIVRTAIEAMAAVFGGTQSLHTNSFDEALGL | |
| | PTVKSARIARNTQIIIQEESGIPKVADPWGGSYM | |
| | MECLTNDVYDAALKLINEIEEMGGMAKAVAEGI | |
| | PKLRIEECAARRQARIDSGSEVIVGVNKYQLEKE | |
| | DAVEVLAIDNTSVRNRQIEKLKKIKSSRDQALA | |
| | ERCLAALTECAASGDGNILALAVDASRARCTVG | |
| | EITDALKKVFGEHKANDRMVSGAYRQEFGESKE | |
| | ITSAIKRVHKFMEREGRRPRLLVAKMGQDGHDR | |
| | GAKVIATGFADLGFDVDIGPLFQTPREVAQQAV | |
| | DADVHAVGISTLAAGHKTLVPELIKELNSLGRPD | |
| | ILVMCGGVIPPQDYEFLFEVGVSNVFGPGTRIPK | |
| | AAVQVLDDIEKCLEKKQQSV | |
| 24 | MGSSHHHHHHGSSMKIEEGKLVIWINGDKGYN | MBP-TAT- |
| | GLAEVGKKFEKDTGIKVTVEHPDKLEEKFPQVA | MTSmcm-MCM |
| | ATGDGPDIIFWAHDRFGGYAQSGLLAEITPDKAF | |
| | QDKLYPFTWDAVRYNGKLIAYPIAVEALSLIYN | |
| | KDLLPNPPKTWEEIPALDKELKAKGKSALMFNL | |
| | QEPYFTWPLIAADGGYAFKYENGKYDIKDVGV | |
| | DNAGAKAGLTFLVDLIKNKHMNADTDYSIAEA | |
| | AFNKGETAMTINGPWAWSNIDTSKVNYGVTVL | |
| | PTFKGQPSKPFVGVLSAGINAASPNKELAKEFLE | |
| | NYLLTDEGLEAVNKDKPLGAVALKSYEEELAK | |
| | DPRIAATMENAQKGEIMPNIPQMSAFWYAVRTA | |
| | VINAASGRQTVDEALKDAQTNGIEENLYFQSNI | |
| | RKKRRQRRRGSDPNSSSLRAKNQLFLLSPHYLR | |
| | QVKESSGSRLIQQRLLHQQQPLHPEWAALAKKQ | |
| | LKGKNPEDLIWHTPEGISIKPLYSKRDTMDLPEE | |
| | LPGVKPFTRGPYPTMYTFRPWTIRQYAGFSTVEE | |
| | SNKFYKDNIKAGQQGLSVAFDLATHRGYDSDNP | |
| | RVRGDVGMAGVAIDTVEDTKILFDGIPLEKMSV | |
| | SMTMNGAVIPVLANFIVTGEEQGVPKEKLTGTIQ | |
| | NDILKEFMVRNTYIFPPEPSMKIIADIFEYTAKHM | |
| | PKFNSISISGYHMQEAGADAILELAYTLADGLEY | |
| | SRTGLQAGLTIDEFAPRLSFFWGIGMNFYMEIAK | |
| | MRAGRRLWAHLIEKMFQPKNSKSLLLRAHCQTS | |
| | GWSLTEQDPYNNIVRTAIEAMAAVFGGTQSLHT | |
| | NSFDEALGLPTVKSARIARNTQIIIQEESGIPKVA | |
| | DPWGGSYMMECLTNDVYDAALKLINEIEEMGG | |
| | MAKAVAEGIPKLRIEECAARRQARIDSGSEVIVG | |
| | VNKYQLEKEDAVEVLAIDNTSVRNRQIEKLKKI | |
| | KSSRDQALAERCLAALTECAASGDGNILALAVD | |
| | ASRARCTVGEITDALKKVFGEHKANDRMVSGA | |
| | YRQEFGESKEITSAIKRVHKFMEREGRRPRLLVA | |
| | KMGQDGHDRGAKVIATGFADLGFDVDIGPLFQT | |
| | PREVAQQAVDADVHAVGISTLAAGHKTLVPELI | |
| | KELNSLGRPDILVMCGGVIPPQDYEFLFEVGVSN | |
| | VFGPGTRIPKAAVQVLDDIEKCLEKKQQSV | |
| 25 | MGSSHHHHHHSSGLVPRGSHMRKKRRQRRRGS | TAT-ΔMTS- |
| | DPNSSSLHQQQPLHPEWAALAKKQLKGKNPEDL | MCM |
| | IWHTPEGISIKPLYSKRDTMDLPEELPGVKPFTRG | |
| | PYPTMYTFRPWTIRQYAGFSTVEESNKFYKDNIK | |
| | AGQQGLSVAFDLATHRGYDSDNPRVRGDVGMA | |
| | GVAIDTVEDTKILFDGIPLEKMSVSMTMNGAVIP | |
| | VLANFIVTGEEQGVPKEKLTGTIQNDILKEFMVR | |
| | NTYIFPPEPSMKIIADIFEYTAKHMPKFNSISISGY | |
| | HMQEAGADAILELAYTLADGLEYSRTGLQAGLT | |
| | IDEFAPRLSFFWGIGMNFYMEIAKMRAGRRLWA | |
| | HLIEKMFQPKNSKSLLLRAHCQTSGWSLTEQDP | |
| | YNNIVRTAIEAMAAVFGGTQSLHTNSFDEALGL | |
| | PTVKSARIARNTQIIIQEESGIPKVADPWGGSYM | |
| | MECLTNDVYDAALKLINEIEEMGGMAKAVAEGI | |
| | PKLRIEECAARRQARIDSGSEVIVGVNKYQLEKE | |
| | DAVEVLAIDNTSVRNRQIEKLKKIKSSRDQALAE | |
| | RCLAALTECAASGDGNILALAVDASRARCTVGE | |
| | ITDALKKVFGEHKANDRMVSGAYRQEFGESKEI | |
| | TSAIKRVHKFMEREGRRPRLLVAKMGQDGHDR | |
| | GAKVIATGFADLGFDVDIGPLFQTPREVAQQAV | |
| | DADVHAVGISTLAAGHKTLVPELIKELNSLGRPD | |
| | ILVMCGGVIPPQDYEFLFEVGVSNVFGPGTRIPK | |
| | AAVQVLDDIEKCLEKKQQSV | |

### Protein Expression

E. coli BL21-CodonPlus (2DE3) or Rosseta competent cells, transformed with plasmids encoding the fusion proteins TAT-MTS-MCM fusion proteins described herein above were incubated at 37° C. in a saline lactose broth (SLB medium) containing kanamycine (50 μg/ml), tetracycline (12.5 μg/ml) and chloramphenicol (34 μg/ml). At an O.D.₆₀₀ of 0.2-0.3, 0.1% glycerol and 0.1 mM potassium glutamate were added to the culture which was then subjected to a heat-shock for 20-30 min at 42° C., after which the bacteria were grown at 37° C. until an O.D.₆₀₀ of 0.8. Protein expression was induced by adding isopropyl-beta-D-thiogalactopyranoside (IPTG, for final concentrations see Table 1 below). After 18 hours of incubation at 12° C., the cells were harvested by centrifugation (2000 g for 20 minutes at 4° C.). Table 2 below summarizes the expression conditions of TAT-MTS-MCM fusion proteins.

TABLE 2
Growth conditions and concentration
of the fusion protein constructs.

| | | IPTG | Growth | Final protein conc. |
|---|---|---|---|---|
| MTS | Host | (mM) | conditions | (mg/ml) |
| MCM | Codon+ | 0.05 | 12° C., O.N. | 0.73 |
| CS | Codon+ | 0.05 | 12° C., O.N. | 2.7 |
| LAD | Rosseta | 0.05 | 12° C., O.N. | 0.43 |
| ΔMTS | Rosseta | 0.05 | 12° C., O.N. | 0.75 |
| MCM, MBP tag | Rosseta | 0.05 | 12° C., O.N. | 0.56 |

### Protein Purification

For the purification procedure, bacterial pellets from 4 liter culture of expressing cells were disrupted using a Microfluidizer (Microfluidics) in binding buffer (25 mM TrisHCl pH8.0, 0.2M NaCl, 10% glycerol, 5 mM betamercaptoEthanol, 1 mM phenylmethylsulphonylfluoride (PMSF)) containing 0.2 mg/ml lysozyme. The suspensions were clarified by centrifugation (24,000 g for 1 h at 4° C.), and imidazole (Sigma Aldrich, St. Louis, Mo., USA)) was added to a final concentration of 10 mM. The supernatants containing the fusion proteins were loaded onto pre-equilibrated (in binding buffer) HiTrap Chelating HP columns (Amersham-Pharmacia Biotech, Uppsala, Sweden). Columns were washed by stepwise addition of increasing imidazole concentrations. Finally, the target proteins were eluted with elution buffer (binding buffer, 250 mM imidazole). All purification procedures were carried out using the FPLC system AKTA (Amersham-Pharmacia Biotech). Imidazole was removed by transferring the purified proteins to PBS using PD-10 desalting columns (GE Healthcare, Piscataway, N.J., USA). Aliquots of the proteins were kept frozen at −80° C. until use.

### Determination of Protein Concentration

Protein concentration was measured according to the Bradford method, using the Bradford reagent and a standard curve of BSA. Protein concentration was determined at a wavelength of 595 nm.

### Separation of Proteins by Electrophoresis

Samples from the various protein fractions (5-20 μg protein/lane) were loaded on 12% (w/v) SDS/PAGE gels.

### Western Blot Analysis

Samples were separated on 12% SDS-PAGE gels. The proteins were then electro-transferred onto Immobilon-P transfer membrane (Millipore, Millipore, Bradford, Mass., USA). Western blot analysis was performed using either anti-MCM (Abcam, Mass., USA), or anti-His (Amersham-Pharmacia Biotech) antibodies at dilutions of 1:1000 and 1:30,000 respectively, to identify the relevant proteins. Band visualization was done using an enhanced chemiluminescence kit (EZ-ECL, Biological Industries, Beit-Haemek, Israel.

### Isolation of Mitochondria

Mitochondria were isolated using a differential centrifugation. Cells were homogenized in buffer A (320 mmol/L sucrose, 5 mmol/L Tris-HCl, 2 mmol/L EGTA, pH 7.4) and centrifuged for 3 min at 2,000 g to remove nuclei and cell debris. The supernatant obtained was centrifuged for 10 min at 12,000 g at 4° C. to pellet the mitochondria. The mitochondrial pellet was washed again twice with buffer A and kept at −80° C. until use.

Delivery of the Fusion Protein into Cells

Cells were plated on 3 T-75 flasks. When the cells reached 90% confluence, the medium was replaced with fresh medium containing 0.02-0.05 μg/μl (final concentration) TAT-MTS-MCM fusion protein constructs, for various time periods. After incubation, the cells were washed with phosphate-buffered saline (PBS), trypsinized, pelleted and kept at −80° C. until use. The pellets were re-suspended in cell lysis buffer (Promega) containing 1 mmol/L PMSF, kept on ice for 10 min and centrifuged at 15,000 g for 10 min. The supernatants were analyzed by Western blot.

### Measurement of ATP Levels

Cells were cultured for 48 hours in a glucose-free medium which contained DMEM (without D-Glucose, Sodium Pyruvate and L-Glutamine), 10% Certified Foetal Bovine Serum (FBS) Dialyzed, 2 mM L-glutamine, 100 U/mL penicillin, and 100 μg/mL, (Biological Industries, Beit Ha'emek, Israel), 1.25 μM Vitamin B-12, which is an essential cofactor of MCM and 5 mM galactose (Sigma).

Mitochondrial ATP levels were determined 6 hours following incubation with 1 μg of each of the four fusion protein constructs. ATP levels were measured using the ATPLite luminescence-based assay according to the manufacturer's instructions (Perkin Elmer, Waltham, Mass., USA) and are expressed as levels relative to control patients' cells, i.e. not treated with any of the fusion protein constructs (PBS only added).

### Mitochondrial Membrane Potential

Mitochondrial content and mitochondrial membrane potential were estimated using, respectively, MitoTracker Green FM (MTG) (Molecular Probes, Eugene, Oreg., USA) and tetramethylrhodamine ethyl ester (TMRE) (Abcam, Mass, USA). MTG was added to the existing medium to a final concentration of 200 nM and the cells were incubated for 45 minutes at 37° C., 5% CO₂. TMRE was added successively to the existing medium to a final concentration of 200 nM and the cells were incubated for an additional 20 minutes at 37° C., 5% CO₂. Medium was removed and, after rinsing once with PBS for MTG or 0.2% BSA in PBS for TMRE, replaced with 100 μl PBS. The plate was read at 37 ° C., λex 485 nm, λem 528 nm (MTG) and λex 485 nm, λem 590 nm (TMRE).

### Oxygen Consumption

Oxygen consumption rate (OCR) was measured using an XF24 extracellular flux analyzer (Seahorse Biosciences, North Billeric, Mass., USA).

### Cell Viability Tests

Cells were plated in a 96 well plate (15,000 cells per well) in 100 μl of glucose-free medium for 24 hours. The following day, 1.5μg of the fusion proteins were added for 72 hours. Mitochondrial isolation buffer alone was used as control. Cell viability was assayed using CellTiter-Blue® (Promega, Madison, Wis.) a fluorescence-based assay, according to the manufacturer's manual.

### Example 2

### Construction, Expression and Purification of the TAT-MTS-MCM Fusion Protein Constructs

In order to test the feasibility of efficiently targeting the mitochondria with fusion proteins based on methylmalonyl-CoA mutase (MCM), MCM-based fusion protein constructs comprising a transactivator of transcription (TAT) domain and various different mitochondrial translocating sequences (MTSs) were prepared and analyzed, as detailed above.

The MTSs used were either the homologous, native MTS of MCM, or heterologous MTSs of human, nuclear-encoded mitochondrial proteins that are classical MTS sequences to target the human MCM protein into the mitochondria. The heterologous MTSs used were of lipoamide deydrogenase (also referred to herein as “lad” or “LAD” having the amino acid sequence denoted by SEQ ID NO. 6), the respective fusion protein construct is referred to herein as “TAT-MTSTlad-MCM”, of citrate synthase (cs, having the amino acid sequence denoted by SEQ ID NO. 4), the respective fusion protein construct is referred to herein as “TAT-MTScs-MCM”, and the native MTS of MCM (mcm, having the amino acid sequence denoted by SEQ ID NO. 5), the respective fusion protein construct is referred to herein as “TAT-MTSmcmMCM”.

Furthermore, a fusion protein construct lacking an MTS, referred to herein as “TAT-AMTS-MCM” was prepared.

All of the plasmids were cloned with a His-tag at the 5′-termini of their coding sequence to facilitate their purification and all of the coding sequences were under the control of the T7 promotor.

A schematic presentation of the various fusion protein constructs is shown in FIG. 1 and the sequences of the various fusion protein constructs prepared as described above are detailed in Table 1. As shown in Table 1, two fusion protein constructs comprising an MTS of citrate synthase (cs) were prepared, “TAT-MTScs-MCM” and “TAT-MTScs-MCM variant” and their amino acid sequences are denoted by SEQ ID NO: 18 and SEQ ID NO: 19, respectively.

The fusion protein construct denoted by SEQ ID NO: 18 (TAT-MTScs-MCM) comprises from its N to C termini a linker having the amino acid sequence MGSS (denoted by SEQ ID NO: 9), a histidine tag (having the amino acid sequence HHHHHH, denoted by SEQ ID NO: 1), an additional linker having the amino acid sequence SSGLVPRGSHM (denoted by SEQ ID NO: 10), a TAT domain (having the amino acid sequence RKKRRQRRR, denoted by SEQ ID NO: 3), a further linker having the amino acid sequence GSDP (denoted by SEQ ID NO: 12), the MTS of citrate synthase (denoted by SEQ ID NO: 4), an additional linker situated between the MTS and MCM having the amino acid sequence of GSS (denoted by SEQ ID NO: 14) and the MCM protein (denoted by SEQ ID NO: 8).

The fusion protein construct denoted by SEQ ID NO: 19 (TAT-MTScs-MCM variant) does not comprise the linker GSS, as evident from Table 1, and therefore the MTS and MCM fragments of this fusion protein construct are directly connected.

As also shown in Table 1, two fusion protein constructs comprising an MTS of Methylmalonyl Coenzyme A Mutase (MCM) were prepared and their amino acid sequences are denoted by SEQ ID NO: 20 and SEQ ID NO: 21 and two fusion protein constructs comprising an MTS of lipoamide deydrogenase (lad) were prepared and their amino acid sequences are denoted by SEQ ID NO: 22 and SEQ ID NO: 23.

In addition to the above His containing fusion protein constructs, a fusion protein construct in which the His-Tag can be removed from the final product was also prepared and is termed herein “TAT-MTSmbp-MCM” or MBP-TAT-MTSmcm-MCM, the amino acid sequence of which is denoted by SEQ ID NO: 24. This fusion protein construct comprises (from its N to C termini) a His and a maltose binding protein (MBP) tags, followed by the TEV protein cleavage site, a TAT domain, the native MTS of MCM and MCM.

A fusion protein construct lacking the MTS was also prepared and is termed herein “His-TAT-AMTS-MCM”, the amino acid sequence of which is denoted by SEQ ID NO: 25. Clones were confirmed by restriction enzymes and sequencing analyses.

Expression of the fusion protein constructs was performed in E. coli hosts. The expression host and conditions for expression were calibrated for each one of the TAT fusion protein constructs individually, by changing several parameters, including the host (which was selected from BL21, BL21 codon plus, Rosseta and HMS the concentration of the inducer (IPTG) and length and conditions of induction period (namely temperature, addition of chemicals, etc., as indicated in Table 1 above).

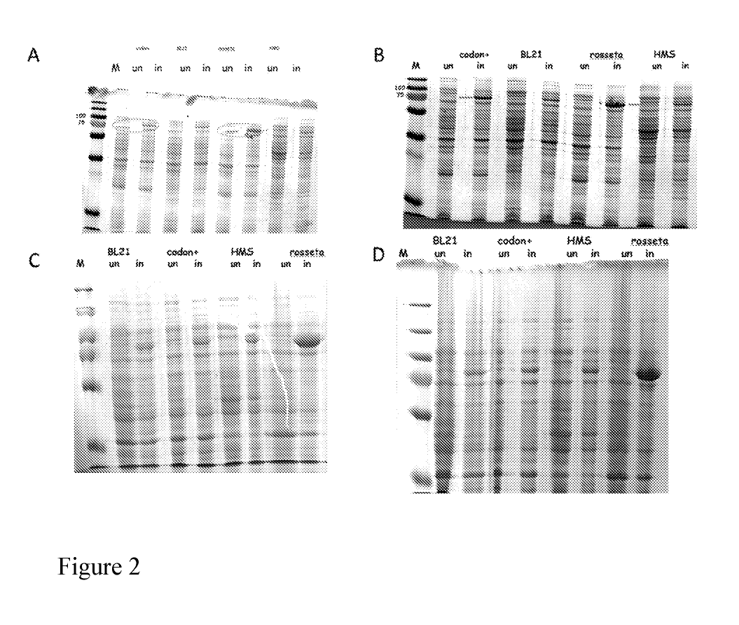

Codon+ bacterial cells were chosen for expression of the fusion protein constructs TAT-MTScs-MCM and TAT-MTSmcmMCM, while rosseta bacteria cells were chosen for the fusion protein constructs TAT-MTSTlad-MCM and TAT-AMTS-MCM, since expression in these cells appeared most efficient as shown in FIG. 2.

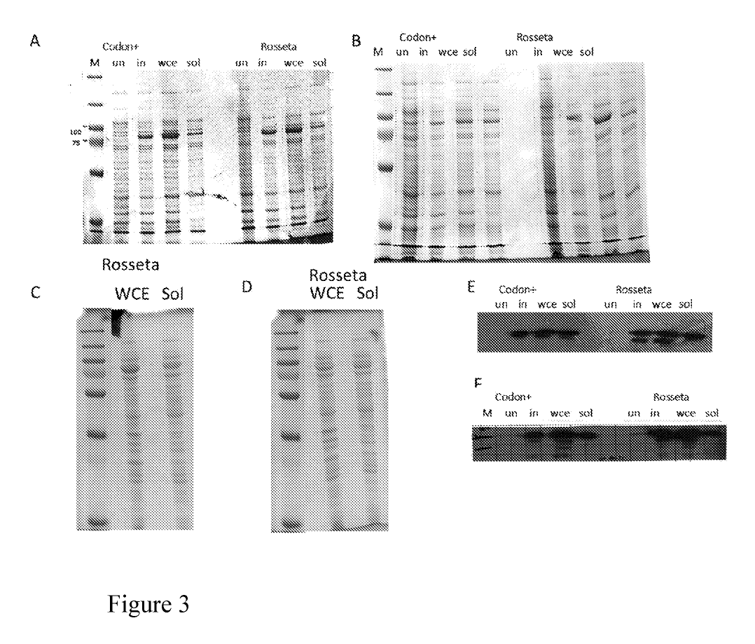

Upon expression, bacterial cells were disrupted and cellular sub-fractions were prepared, separating the soluble and insoluble fractions, as described above. As shown in FIG. 3, analysis was performed for the whole cell bacteria (wce), the soluble fraction (sol), and insoluble fraction on SDS-PAGE gels in order to examine whether the fusion protein was expressed and at which sub-cellular fraction it accumulated. The goal was to obtain high expression levels of the different TAT-fusion proteins in the soluble sub-fraction of the expressing bacteria, for future purification. The different TAT-fusion protein constructs were also characterized by Western blots analyses using anti-His antibody, as shown in FIG. 3E and FIG. 3F.

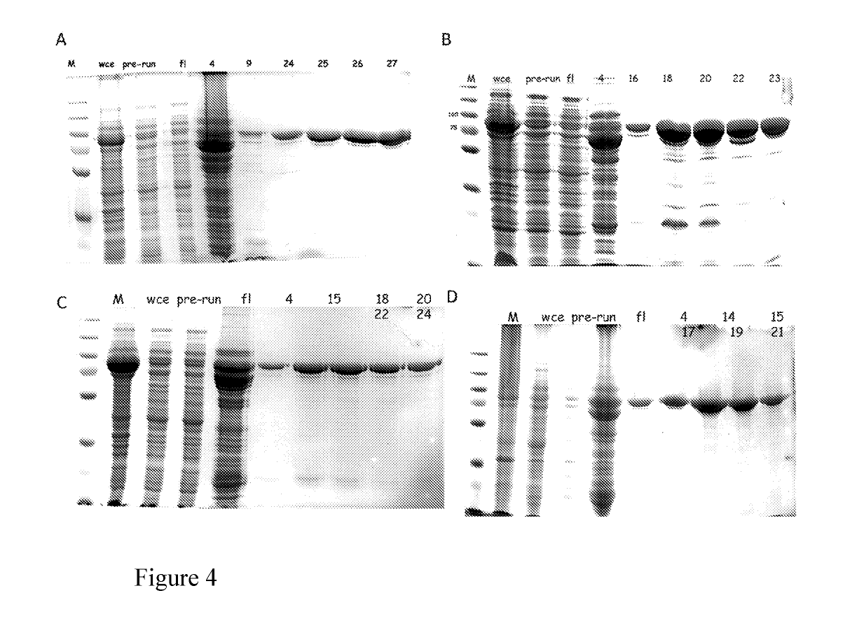

Next, the various fusion protein constructs were highly purified using affinity chromatography, as described above. The soluble fraction of each one of the fusion protein constructs, each comprising a different MTS sequence, was loaded on a Ni-chelating column, followed by multiple washing steps with increasing concentrations of imidazole and finally eluted at a high concentration of imidazole, as demonstrated in FIG. 4A-FIG. 4D.

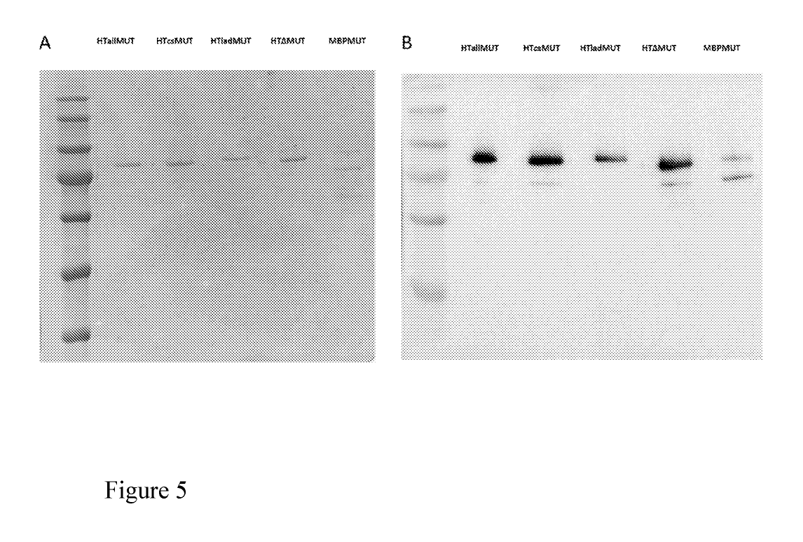

In order to characterize the fusion proteins, the eluted proteins were analyzed by SDS-PAGE and Western blot analysis, using anti-MCM antibodies. As shown in FIG. 5A and FIG. 5B, purified fusion protein constructs showed a major band at the expected size (approximately 87 kDa). A slight size variation was observed among the four fusion protein constructs, resulting from the variation in length of the various MTS polypeptides used.

EXAMPLE 3

Internalization of TAT-MTS-MCM Fusion Protein Constructs and their Processing

In order to test the ability of the various TAT-MTS-MCM fusion protein constructs to reach the mitochondria within intact cells, patient 673 fibroblasts, carrying a stop codon mutation in the gene encoding for the methylmalonyl-CoA mutase (MCM) protein, were first incubated for 24 hours with various concentration of TAT-MTScs-MCM (having the amino acid denoted by SEQ ID NO: 18) and cell internalization (into whole cell lysates) was determined by Western blot analysis. As shown in FIG. 6A, TAT-MTScs-MCM successfully internalized into the cells.

The ability of additional fusion protein constructs to internalize into mitochondria was also examined as detailed below. After incubation of patient 673 fibroblasts in the presence of the various fusion protein constructs, sub-cellular fractions were prepared, in order to separate the mitochondria from the cytosol. Mitochondrial samples were then analyzed by Western blot for the presence of the MCM-based fusion protein constructs using anti-His antibodies (FIG. 6B) and anti-MCM antibodies (FIG. 6C and FIG. 6D).

As shown in FIG. 6, Western Blot analysis confirmed the internalization of the fusion protein constructs within the mitochondria.

Moreover, by using anti-MCM antibodies (FIG. 6C and FIG. 6D) it was demonstrated that the TAT-MTScs-MCM, TAT-MTSlad-MCM as well as the TAT-MTSmcm-MCM fusion protein constructs underwent processing, as evident by the appearance of the additional product, smaller in its size that reacted with the specific antibodies (FIG. 6C, marked as processed protein).

However, the TAT-AMTS-MCM fusion protein construct the lacks any MTS, although reaching the mitochondria most probably due to the TAT sequence the allows crossing of biological membranes, was the only fusion protein construct that did not undergo any processing, as evident from FIG. 6D.

### Example 4

### Effect of Mitochondrial Targeted MCM on Mitochondrial ATP Production.

In order to determine whether the mitochondrial-targeted MCM fusion protein constructs are able to affect ATP produced by oxidative phosphorylation (OXPHOS) in the mitochondria, GM01673, GM00050 and 346 fibroblasts were cultured for 48 hours in a glucose-free, OXPHOS dependent medium supplemented with dialyzed serum as an energy source, and 1.25 μM Vitamin B-12 which is an essential cofactor of MCM.

Mitochondrial ATP levels were determined 6 hours after incubation with 10 μg/ml (100 μl volume) of each one of the mitochondrial targeted MCM fusion protein constructs (namely, TAT-MTScs-MCM, TAT-MTSTlad-MCM, TAT-AMTS-MCM, TAT-MTSmbp-MCM and TAT-MTSmcmMCM, denoted by SEQ ID NO: 18, SEQ ID NO: 22, SEQ ID NO: 25, SEQ ID NO: 24 and SEQ ID NO: 20, respectively.

As shown in FIG. 7, a significant enhancement of between 14-21% in ATP levels was observed in 346 cells (FIG. 7A) with all of the fusion protein constructs, while TAT-MTScs-MCM and TAT-AMTS-MCM resulted in the largest increase.

In GM01673 fibroblasts (FIG. 7B) an increase of 15-25% was observed in ATP levels upon treatment with the various fusion protein constructs, while the fusion protein construct TAT-MTScs-MCM resulted in the largest increase. Interestingly, the fusion protein construct TAT-AMTS-MCM did not affect ATP levels relative to the control in these cells, implying that its processing is important for MCM activity. GM00050 fibroblasts resulted in no significant change in ATP levels relative to the control (FIG. 7C).

### Example 5

### Effect of Mitochondrial Targeted MCM Fusion Protein Constructs on Mitochondrial Membrane Potential

In order to determine whether mitochondrial targeted MCM could affect mitochondrial membrane potential, GM01673 fibroblasts were cultured for 24 hours in a glucose-free, OXPHOS dependent medium supplemented with dialyzed serum as an energy source and 1.25 μM Vitamin B-12 was added. Mitochondrial membrane potential was determined 6 hours after incubation with 15 μg/ml (100 μl volume) of mitochondrial targeted MCM fusion protein constructs (TAT-MTScs-MCM and TATmcmMCM denoted by SEQ ID NO: 18 and SEQ ID NO: 20, respectively).

As described above, 1 hour before the end of incubation time (namely after 5 hours) MitoTracker Green FM was added to the medium to a final concentration of 200 nM. Then, 30 minutes before the end of the incubation time FCCP (20 μM) was added as a control and 20 minutes before the end of the incubation time TMRE was added at 200 nM. TMRE wells were washed once with PBS supplemented with 0.2% BSA and suspended in an additional 100 μl. MTG wells were washed once with and suspended in an additional 100 μl. The TMRE/MTG ratio was measured as described above.

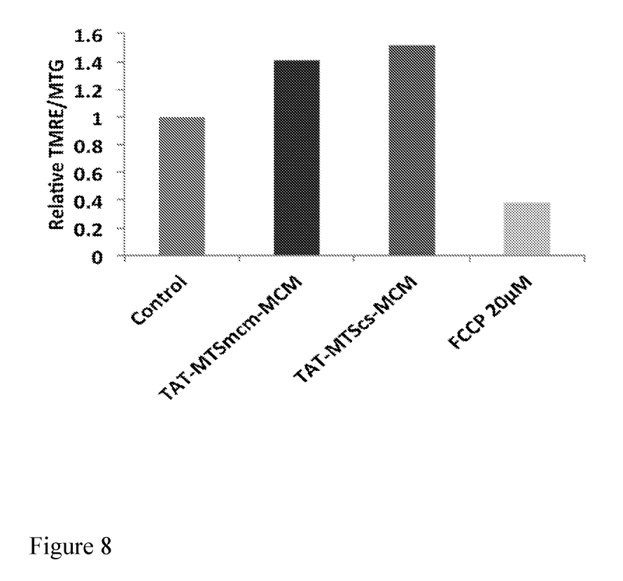

As shown in FIG. 8, both fusion protein constructs changed the mitochondrial membrane potential, where the construct TAT-MTScs-MCM resulted in a slightly higher change in mitochondrial membrane potential (51%) than TATmcmMCM (40%).

### Example 6

### Effect of Mitochondrial Targeted MCM Fusion Protein Constructs on Oxygen Consumption

In order to determine whether mitochondrial targeted MCM fusion protein constructs are able to affect oxygen consumption by the mitochondria, which is an additional marker of mitochondrial activity, GM01673 fibroblasts were cultured for 48 hours in a glucose-free, OXPHOS dependent medium supplemented with dialyzed serum as an energy source and 1.25 μM Vitamin B-12.

Oxygen consumption was determined using Seahorse Extracellular Flux (XF) Analyzer 6 hours after incubation with 10 μg/ml (100 μl volume) of each one of the mitochondrial targeted MCM fusion protein constructs. As shown in FIG. 9, a significant enhancement of 50-102% (relative to the control) was observed in oxygen consumption upon treatment with the various fusion protein constructs. TAT-MTScs-MCM resulted in the largest increase in oxygen consumption, while again TAT-ΔMTS-MCM showed reduced activity.

### Example 7

### Effect of Mitochondrial Targeted MCM Fusion Proteins on Cell Viability

In order to determine whether delivery of TAT-MTS-MCM fusion proteins into MMA patient cells could affect cell viability, GM01673, GM00050 or 346 patient fibroblasts were cultured for 24 h in an OXPHOS dependent medium. Cell viability was determined 72 h after incubation with 15 μg/ml of each fusion protein. As shown in FIG. 10 a significant increase of 13-30% in cell viability was observed in GM01673 (FIG. 10A), 11-36% in GM00050 (FIG. 10B) and 12-22% in 346 (FIG. 10C) fibroblasts with all fusion proteins. Again treatment with TAT-MTScs-MCM fusion protein resulted in the highest improvement (27%) in cell viability, whereas TAT-MTSA-MCM treatment showed reduced activity in GM01673 cells as compared to untreated control cells.

### Example 8

### Effect of Mitochondrial Targeted MCM Fusion Proteins on MMA Levels.

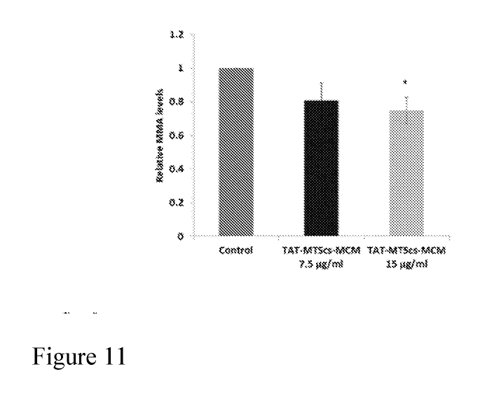

The major symptom of MMA pathology is elevated MMA levels, which may account for multisystem pathological effects [12, 13]. In order to determine whether delivery of TAT-MTS-MCM fusion proteins into MMA patient cells could reduce MMA levels, GM01673 patient fibroblasts were cultured for 24 h in an OXPHOS dependent medium. Methylmalonic acid levels were determined in whole cell lysates using an ELIZA kit 48 h after incubation with 7.5 or 15 μg/ml of TAT-MTScs-MCM. As shown in FIG. 11 a 25% significant reduction in MMA levels was observed after treatment with 15 μg/ml of TAT-MTScs-MCM.

Interestingly, other reported treatments [14] based on gene therapy using an adeno-associated virus serotype 8 vector (AAV8) also show that the levels of MMA aren't thoroughly reduced as expected (−40% reduction observed) [15]. This is consistent even if the AAV8 is delivered to newborn mice and even after readministration of the virus [16]. Therefore, the mitochondria may be already irreversibly damaged at the time of the treatment. Thus, supplying the treatment at an earlier stage, for example during pregnancy, should be considered.

### Example 9

### Effect of TAT-MTS-MCM Fusion Proteins on Liver Secretion

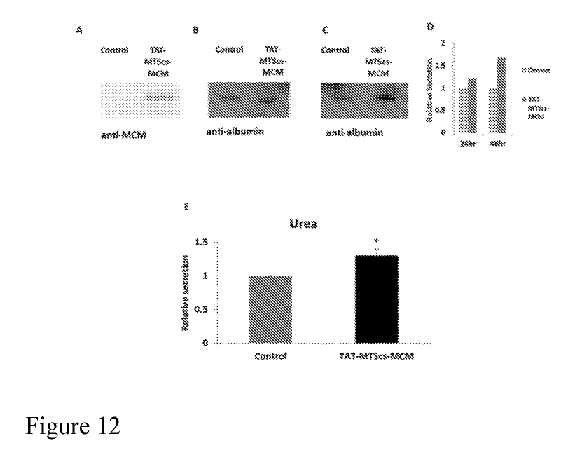

The effects of TAT-MTS-MCM fusion proteins were all tested in cultures of fibroblast obtained from MMA patients. However, since the main organ affected in MMA pathology is the liver [16], we aimed to produce a liver cell line with lacks MCM expression to mimic MMA liver pathology. Recently, Erlich et al have shown that mitochondrial function is essential for secretion of mediators from mast cells [17]. Therefore, our hypothesis was that secretion in MCM mutated liver cells could be impaired. To check this hypothesis we knocked out the MUT gene from HepG2 cells, an accepted model for albumin secretion [18], using the CRISPR technology. Deletion of the MUT gene was confirmed by Western blot analysis with anti-MCM antibodies (FIG. 12A). MCM protein levels were not detected in HepG2 MCM (−/−) cells. Next, the levels of albumin were determined by Western blot analysis in the growth medium of the HepG2 mut(−/−) cells (OXPHOS medium) treated for 24 h (FIG. 12B) or 48 h (FIG. 12C) with 15 μg/ml of TAT-MTScs-MCM, compared to control. As shown in FIG. 12B-C the levels of secreted albumin were increased following treatment with TAT-MTScs-MCM (21% for 24 h, 69% for 48 h). In addition, it is well established that urea is secreted from liver cells. Therefore, the levels of urea in the growth medium were also determined by Covas analysis after treatment for 48 h with TAT-MTScs-MCM fusion protein (samples were normalized to the protein concentration of the lyzed cells). As shown in FIG. 12C the levels of secreted urea were increased by 29% following treatment with TAT-MTScs-MCM. To conclude restoring MCM activity in liver cells may affect major functions of the liver such as albumin and urea secretion. Moreover, we suggest a role for mitochondrial function and MCM activity in secretion of mediators from the liver.

### Example 9

### Effect of TAT-MTS-MCM Fusion Proteins Cross the Placenta In Vivo in Pregnant Mice

Given TAT's ability to cross many different cell and tissue types we hypothesized that TAT-MTS-MCM would be able to cross the placenta. We tested the ability and efficacy of TAT-MTS-MCM proteins to cross the placenta and deliver to the embryos in mouse model, a requirement which is also crucial for future human treatment.

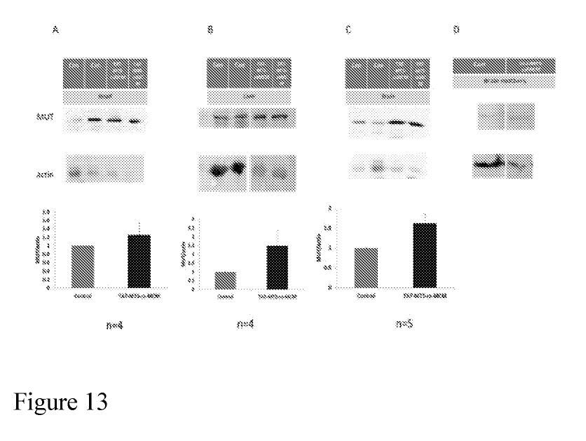

Since in the mouse model of methyl malonyl Co-mutase mortality is observed at the day of birth, we injected W.T C57BL mice in our treatment at the last week of pregnancy three times, the presence of the enzyme examined in several tissues by western blot analysis using anti-Methyl malonyl Co-A mutase and anti-β actin antibodies. We chose to work with the three major organs that have the highest energy demands, and so are usually affected the most in mitochondrial disorders—the liver, the heart (muscles), and the brain. As can be seen in FIGS. 13A-D, amounts of TAT-MTScs-MCM protein was significantly higher in the heart (A), as well as in the liver (B) but, most importantly, in the brain (A), of the new pups in treated compered to control mice, demonstrating the delivery of TAT-MTScs-MCM into the tissues furthermore, the amount of the TAT-MTScs-MCM is higher in the brain's mother that injected in the treatment than the control.

We also measured the ratio MUT/actin and as we can see the amount of the protein was higher in the tissues of treated mice compared to control mice.

### REFERENCES

References considered to be relevant as background to the presently disclosed subject matter are listed below:

Jansen, R. and Ledley, F. D. 1990, Am. J. Hum. Genet., 47: 808-814.

Van't Hoff, W. G. et al., 1998, J. Pediat., 132: 1043-1044.

Nyhan, W. L. et al., 2002, Eur. J. Pediat., 161: 377-379.

Oberholzer, V. G. et al., 1967, Arch. Dis. Child, 42: 492-504.

Walter, J. H. et al., 1989, Eur. J. Pediat., 148: 344-348.

Senac, J. S. et al., 2012, Gene Ther., 19(4): 385-391.

Rapoport M. et al., 2008, Mol. Ther., 16: 691-697.

WO 2009/098682.

WO 2014/170896.

Fowler, B. et al. 2008, J. Inherit. Metab. Dis., 31: 350-360.

Worgan, L. C. et al. 2006, Hum. Mutat., 27: 31-43.

Kanaumi, T., et al., Pediat. Neurology, 2006. 34(2): p. 156-159.

Mesa-Medina, O., et al., Nefrologia, 2014. 34(4): p. 539-540.

Caterino, M., et al., Mol. BioSys., 2016. 12(2): p. 566-74.

Chandler, R. J. and C. P. Venditti, Mol. Ther., 2010. 18(1): p. 11-16.

Niemi, A. K., et al., J. Pediatr. 2015. 166(6): p. 1455-61 el.

Erlich, T. H., et al., J Allergy Clin Immunol., 2014. 134(2): p. 460-9.

Ha, C. E., et al., J Biomed Sci., 2009. 16: p. 32.

Acknowledgement of the above references herein is not to be inferred as meaning that these are in any way relevant to the patentability of the presently disclosed subject matter.

1. A fusion protein comprising a HIV-1 transactivator of transcription (TAT) domain, a functional human Methylmalonyl Coenzyme A mutase (MCM) and a human mitochondria targeting sequence (MTS) situated between said TAT domain and said functional human MCM.

2. The fusion protein according to claim 1, wherein said functional human MCM is C-terminal to said human MTS.

3. The fusion protein according to claim 1, wherein said human MTS is the MTS of human MCM or heterologous to said human MCM.

4. The fusion protein according to claim 1, wherein said human MTS is human mitochondrial citrate synthase MTS, having the amino acid sequence denoted by SEQ ID NO: 4 or human lipoamide dehydrogenase MTS, having the amino acid sequence denoted by SEQ ID NO: 6.

5. The fusion protein according to claim 4, wherein said human MTS is human citrate synthase MTS having the amino acid sequence denoted by SEQ ID NO: 4.

6. The fusion protein according to claim 1, wherein said human MTS is human Methylmalonyl Coenzyme A mutase MTS, having the amino acid sequence denoted by SEQ ID NO: 5.

7. The fusion protein according to claim 1, further comprising at least one linker.

8. The fusion protein according to claim 1, wherein said MTS is linked to said functional MCM and/or to said TAT via a linker.

9. (canceled)

11. A fusion protein comprising an HIV-1 transactivator of transcription (TAT) domain having the amino acid sequence denoted by SEQ ID NO: 3 linked to functional human MCM having the amino acid sequence denoted by SEQ ID NO: 8 and a human mitochondrial citrate synthase MTS having the amino acid sequence denoted by SEQ ID NO: 4, said MTS situated between said TAT domain and said functional human MCM, and wherein said MCM is C-terminal to said MTS.

12. The fusion protein according to claim 11, having the amino acid sequence denoted by SEQ ID NO: 18 or SEQ ID NO: 19.

13. A fusion protein comprising an HIV-1 transactivator of transcription (TAT) domain having the amino acid sequence denoted by SEQ ID NO. 3 linked to functional human MCM having the amino acid sequence denoted by SEQ ID NO. 3 and a human mitochondrial lipoamide dehydrogenase MTS having the amino acid sequence denoted by SEQ ID NO. 6, said MTS situated between said TAT domain and said functional human MCM, and wherein said MCM is C-terminal to said MTS.

14. The fusion protein according to claim 13, having the amino acid sequence denoted by SEQ ID NO: 22 or SEQ ID NO: 23.

15. A composition comprising a physiologically acceptable carrier and as an active ingredient a fusion protein according to claim 1.

16. A pharmaceutical composition comprising a pharmaceutically acceptable carrier and as an active ingredient a fusion protein according to claim 1.

17-22. (canceled)

23. A method for treating or alleviating a disease or disorder associated with a deficiency of MCM or with defective MCM in a subject in need thereof, said method comprising the step of administering to said subject a therapeutically effective amount of the fusion protein according to claim 1, thereby treating or alleviating a disease or disorder associated with a deficiency of MCM or with defective MCM.

24. A method for treating or alleviating MIVIA in a subject in need thereof, said method comprising the step of administering to said subject a therapeutically effective amount of the fusion protein according to claim 1, thereby treating or alleviating methylmalonic acidemia (MMA).

25. The method according to claim 23, wherein said MMA is isolated MMA (OMIM 251000) or Methylmalonic acidemia and homocystinuria (OMIM 277400).

26. The method according to claim 23 any one of claims 23 to 25, wherein said method further comprises administering to said subject an additional therapeutic agent.

27. (canceled)

28. The method according to claim 23, wherein said fusion protein or said pharmaceutical composition is intravenously administered to said subject.

29-31. (canceled)

32. A method for substituting, at least in part, activity of a defective, deficient or non-functional human MCM in a subject in need, comprising administering to said subject a therapeutically effective amount of the fusion protein according to claim 1.
